SEQUENCE LISTING (41 sequences)

SEQ ID NO: 1 - protein, 503 aa, synthetic construct
MVKKTFFTTLAAVSFSAALETALFFSGCGSSLWTTKAHANSVFSSLMQQQGFADIVSQVKPAVVSVQVKSNKKKKEWFFSDFFSTPGFDQLPDQHPLKKFFQDFYNRDKPSNKSLQRSHRLRPIAFGSGFFISSDGYIVTNNHVISDGTSYAVVLDDGTELNAKLIGTDPRTDLAVLKVNEKRKFSYVDFGDDSKLRVGDWVVAIGNPFGLGGTVTAGIVSARGRDIGTGVYDDFIQIDAAVNRGNSGGPTFDLNGKVVGVNTAIFSPSGGNVGIAFAIPAATAKQVVQQLIEKGLVQRGWLGVQIQPVTKEISDSIGLKEAKGALITDPLKGPAAKAGIKAGDVIISVNGEKINDVRDLAKRIANMSPGETVTLGVWKSGKEENIKVKLDSMPEDENMKDGSKYSNEHGNSDETLEDYGLIVAPSDDGVGLVVTDVDPDSDAADKGIRPGDVIVTVNNKSVKKVSDITDTIKNAQKLGRKAILLQVRTNDQNRFVALPIFKK

SEQ ID NO: 2 - protein, 503 aa, synthetic construct
MVKKTFFPTLAAVGFSAALETALFFSGCGSSLWMTEARANSVFASLVQQQGFANIVSQVKPAVVSVQVKSNKKKKEWFFSDFFSSPGLDQLPDQHPLKKFFQEFYDRGKPSNKSLKRPHRLRPIAFGSGFFISSDGYLVTNNHVISDGTSYTVVLDDGTELNAKLIGTDARTDLAVLKVDEKRTFSYVDFGDDSKLRIGDWVVAIGNPFGLGGTVTAGIVSARGRDIGTGVYDDFIQIDAAVNRGNSGGPTFDLNGKVIGVNTAIFSLSGGNVGIAFAIPAAIAKQVVQQLIEKGSVQRGWLGVQIQPVTKEISDSIGLKEAKGALITDPLKGPAAKAGIKAGDVIISVNGEKINDVRDLAKRIANIKPGETVTLGVWKSGKEENIKVKLDTMPDDKVMKEDSKYSNGRNDSNETLEDYGLIVAPSDDGIGLVVTDVDSDSDAADKGIRPGDVIVTVNNKSVKKVSDITDTIKNAQKLGRKAILLQVRTNDQNRFVALPIFKK

SEQ ID NO: 3 - protein, 503 aa, synthetic construct
MVKKTFFKTLIAVSFSAALESALFFSGCGSSLWTTAAHANSVFTSFMQQPGFADIVSQVKPAVVSVQVKSNKKKDEWSFSNFFSGPGFDQLPDQHPLKRFFKEFYDLDKPKNKFPSHSHRLRPIAFGSGFFISSDGYIVTNKHVISDGTSYSVVLDDGTELNAKLIGSDQRTDLAVLKVNDKRKFSYVDFGDDSKLRVGDWVVAIGNPFGLGGTVTAGIVSARGRDIGTGVYDDFIQIDAAVNRGNSGGPTFDLNGKVVGVNTAIFSPSGGNVGIAFAIPAATVKQVVQQLIEKGSVQRGWLGVQIQPVTKEISDSIGLKEDKGALVTDPLKGPAEKAGIKAGDVIISVNGEKINDARDLAKRIANIRPGETVTLGVWRSGKEENIKVKLASMPEDEGKKEGSKYSNKRNDADETLEDYGLIVAPSDDDVGLVVTDVDSDSDAADKGIRPGDVIITVNNKSVKKVSDITDTIKNAQKLGRKAILLQVRTNGQNRFVALPIFKK

SEQ ID NO: 4 - protein, 503 aa, synthetic construct
MLKKTFFKTFVAVSFSAVLESALFFSGCTASLWTTKAHASSVFTSLVQQQGFADIVAQVKPAVVAVQVKSNKKKEDWFFSNFFSGPGIDQLPDQHPLKRLFKEFYDFDKPKNKFPHHSQRLRPIAFGSGFFISSDGYIVTNDHVISEGTSYSVVLDDGTELNAKLIGKDPKTDLAVLKVNDKRKFSYVDFGDDSKLRVGDWVVAIGNPFGLGGTVTAGIVSARGRDIGTSSYDDFIQIDAAVNRGNSGGPTFDLNGKVVGVNTAIFSPSGGNVGIAFAIPAGTAKQVVQQLIEKGSVQRGWLGVMIQPVTKEISDSIGLQEAKGALVTDPLKGPAAKAGIKAGDVIISVNDEKVNDSRDLAKRIANMSPQETVTLGIFRSGKEEKIKVKLAAMPEDEGKKESSKYLNERGNSDETLEDYGLIVAPSEDGLGLVVTDVDSDSDAADKGIRPGDVIVTVNNKSVKKTSDITDAIKNAQKLGRKAILLQVRTNDQNRFVALPIFKK

SEQ ID NO: 5 - protein, 406 aa, synthetic construct
MTTEIRVPTLGESVTEATVGKWFKKLGEAVAVDEPLIELETDKVTVEVPSPVAGKLSEIIAKEGDTVEVKALLGLVEAGAAGISQSFSPSATPIPEVPSELKQSSSSGAMQKDTMPPSPSAAKLMAENNIAKSNISGSGKRGQILKEDVLGVLEQEVKAPSVSAASSSASLVQEKHEERVRMTKLRQTIARRLKDAQNTAAMLTTFNEVDMSAVMDLRKRYKDLFEKKHGVKLGFMGFFTKAVCHALKELPAVNAEIDGTDIVYKNYVNVGIAVGTDKGLVVPVVRHADQMSLAEIEKEIGRLGRLARDGKLAVSDMQGGTFTITNGGVYGSLMSTPILNAPQSGILGMHAIKERAMVVGGQIVIRPMMYLALSYDHRIVDGQEAVTFLVRVKESLEDPERLVLDL

SEQ ID NO: 6 - protein, 410 aa, synthetic construct
MTTGIRVPTLGESVTEATIGKWFKKLGEAVAVDEPLVELETDKVTVEVPSPVMGKLTEIIAKEGDIVEVNAVLGFVESGAAGISQSFSPSATSIPEAPSELEQSPSSSATPSGTMPPAPSAAKLMAENNIAKSDISGSGKRGQILKEDVLGALAQGTKASTSVATLTASSSSAAPIQEMREERVRMTKLRQTIARRLKDAQNTAAMLTTFNEVDMSAVMDLRKRYKDLFEKKHGVKLGFMGFFTKAVCHALKEFPTVNAEIDGTDIVYKNYVNAGIAVGTDKGLVVPVVRDADQMSLAEIEKEISRLGRLARDGKLAVSDMQGGTFTITNGGVYGSLMSTPILNAPQSGILGMHAIKERAMVVGGQIIICPMMYLALSYDHRIVDGQEAVTFLVRVKESLEDPERLVLDL

SEQ ID NO: 7 - protein, 411 aa, synthetic construct
MTTEIRVPTLGESVTEATVGKWFKKLGEAVAIDEPLVELETDKVTVEVPSPVAGKLFEIIAKEGDTVEVNALLGAVEAGAASVAKSPSSSETSVSAAPSELEQSSSSNTMPPAPSAAKLMAENNIAKSDILGSGKRGQILKEDVLNVLAQGVKTSPPAVSASSSTPVSVSSSAVAPVQEMREERVRMTKLRQTIARRLKDAQNTAAMLTTFNEVDMSAVMGLRKRYKDLFEKKHGVKLGFMGFFTKAVCHALKELPAVNAEIDGTDIIYKNYVNAGIAVGTDKGLVVPVVRDADQMSLAEIEKEIGRLGRLARDGKLAVSDMQGGTFTITNGGVYGSLMSTPILNAPQSGILGMHAIKERAMVVDGQIAIRPMMYLALSYDHRIVDGQEAVTFLVRVKESLEDPERLVLDL

SEQ ID NO: 8 - protein, 403 aa, synthetic construct
MTTEIRVPTLGESVTEATVGKWFKKLGEAVAMDEPLVELETDKVTVEVPSPVAGKLSEIIAKEGDTVEVNALLGTVEAGAAGVTQSFSPSATSVPVASSESEKLASSNTMPPSPSAAKLMAENNIAKSDIAGSGKRGQILKEDVLGGLEQKTKTPTPSSSATGSSVVSVPETREERVRMTKLRQTIARRLKDAQNVAAMLTTFNEVDMSAVMDLRKRYKDLFEKKHGVKLGFMGFFTKAVCHALKELPAVNAEIDGTDILYKNYVNVGIAVGTDKGLVVPVVRDADQMSLAEIEKEIGRLGRLARDGKLAVSDMQGGTFTITNGGVYGSLMSTPILNAPQSGILGMHAIKERAMVVEGQVVIRPMMYLALSYDHRIVDGQEAVTFLVRVKESLEDPERLVLDL

SEQ ID NO: 9 - protein, 391 aa, synthetic construct
MCLKVLGNVVRCNQGIALLAMIMVLVGCSSGMQRFANPFSYPTISNQPNISTTISTYTDSPMLQYGAGMIQSTELPPVEPVNDLSTYDNTAYNSPPQDGTSSPNSRIMGTPPRNLGTLSRSQMRNDPLFRQNSYIVQTGDTLLSIARQRGVSVEALKLVNGIRSNSIYIGQVLMIPSGRTAETSNVRNNDWGASKQSLSQSQSASSIRHKKYSSTEKAPITPKPSAQINRSNGEQNSSAQMSSLNYEKGVLDTVMNKDNGTTPQATGISKMRWPVRGRLLSQFGQKKGTTSRGIDIAVPEGSSVKAAENGIVIYASDGLKELGNVVMIRHENNIITIYGCNSKLVVTRGQRIRRGDEIAKSGVSGDVKTPRVYFEVRENSLPVDPIKYLEN

SEQ ID NO: 10 - protein, 391 aa, synthetic construct
MYLKVLGKIFRCNQGITLLAMITILAGCSSGTQRFANIFSHHTVSNRSNMSTTIPTDPGMLSYGGGVIQSTELPPVEPSNDSWADDSAPYNSPRQDGTLSPDGRIIGTPPRNLGTISRSQMNNTPLFRRNSYIVQSGDTLLSIARQIGISVEALKLANGISSNNSIYIGQVLMIPGRRTVATSNAQNDDRVTSTEPSSQSQVRSSIRYQKAPSTDKAPITAMPSSKINRSNAEQDSSTQMPSSNYKTDVLDTVMNTDDGVTPQATGISKMRWPVRGRLLSQFGQKKGTATSRGIDIAVPEGSSVKAAENGIVIYASDGLKELGNVVMIRHENDIITIYGYNSKLVVNRGQRIRRGDEIAKSGVSGDVKTPRVYFEVRKNSLPVDPIEYLEN

SEQ ID NO: 11 - protein, 391 aa, synthetic construct
MCLKVLSNIFWRNFQGIFFLAMVTIFAGCSSGTQRFANIFPHRTESTHSNISTTMSADPQMLPYGGMIQSTELPPVESGSDSWINDNTAYNSTQPRGETSSDSRIMGTPPRDLGTLSRSQMGNSPIFRRNSYIVQSGDTLLSIARQIGINVEALKSANGIRNNSIYIGQVLMIPNSRTLATSNTTNDKRVVSTTETSVQSQNTSAIRYKKASPINKTSATISSSAKINRSNIEKNSSKQMMQLNHEASLPDTITNTDSIVTPQATGISKMRWPVRGRLLSQFGQKKGTATNRGIDIAVPEGSSVKAAENGVVIYASDGLKELGNVVMIRHEDNIITIYGCNSRLVVNKGQRIRRGDEIAKSGISGNVKTPRVYFEVRKNAVPVDPTEYLEN

SEQ ID NO: 12 - protein, 389 aa, synthetic construct
MCLKALDNIFRHNFQRITFLAVVTIVAGCSSGSQRFSNIVSHHRVSSQSNIANSMSSDPGMLSYGGEMIQSTELPPVEPSDDSWIDDNSPQQDRGAPSNGRVMGAPPRNLGTLSRSQMDSSPIFRQNSYIVQSGDTLLSIARQVGVSVEALKSANGINSNSIYIGQVLVLPSRRALATSKVPSYNDLAVSKAESSFQSQMTSSTKHKKASPIYKAPVTTPPSTQVNRSNSEKNSSKQMMQLNHESDLSSTVTRTDNIKTPQATGISKMRWPVRGRLLSQFGQKKGTIMNRGIDIAVPEGSSVKAAENGVVIYAGDGLKELGNVVMIRHENNIITIYGCNSQLVVNKGQRIRRGDEIAKSGVSGNVKTPRVYFEMRKNSLPVDPLKYLEN

SEQ ID NO: 13 - protein, 326 aa, synthetic construct
MSNIYDWSLKADENAHSDNIINWAEGQPPSSVNDSARAMMQRVREYLADNGGSLDSTFIVNAEDKTTSIIVTTASPIEEYKNDIIIRFKAGGVNVGTTTVIVNNIEEKLLYKATNAGVTPLEGGELQRDAIYEMVYNKDVSMEDHGGWYLLNPTPLPPPKIETFPSGFIATFAMQEVPSGWLLCDGAVYERKDYPQLFKAIGDKWGKDSDTTFKVPDFRGMFLRGFDDGRGLDAGRQFADQQHDSIRSHTHIGTIEEAGEHTHKFQYYGVGWNSGDIGRRNPFYYRQSSIGVTQSAGAHTHNISLSSTGEAETRPVNATVVYAIKS

SEQ ID NO: 14 - protein, 323 aa, synthetic construct
MSSIYDWSLEAAENAYADVNINWAEGQPPSSVNNSARVMMQRIKEYLLDNGGVMETQFTINEDKTSIRLITNSPIEAYMDGIVVRFKAQKQNRGITNIALKQLPMQPVYKITKSGIAPLKGQEIQIGGLYELVYICNIDGKNTDGWFLTNPVDKDNFPSGFIGTFAMEKLPDGWLVCDGKEYSRKEYADLFEALGETWGKGDGQTTFNVPDLRGMFLRGLDSGKGIDKKRSLGTKQEESFKAHTHEGTTERAGEHTHKYEVYTKSIDIIASTNGWEAMYNHPNKNKVWETRSAGAHEHKISLKKTGGDETRPVNVAVVYAVKA

SEQ ID NO: 15 - protein, 326 aa, synthetic construct
MSNIYDWSLKADENANSDSIINWAEGQPPSSVNDSARAMMQRVREYLADSGGSIDSSFMVNVEDKTTFITLKTASLIEKYKNDIIIRFKSRGVNIGRTTITVNSIGEKPIYKATNAGIIPLEGGELQTDGIYEIVYNSNVSIEDYDGWFLLNPTPLPPPKVEPFPCGFIATFAMQEMPNGWLLCDGAVYKRKDYPQLFKAIGDKWGKDSNTTFKVPDFRGMFLRGFDNGRGLDSNRQFAKEQQDCIKSHEHVCTIERAGEHTHNFQYDGVGWSANDIGRRNPSYHYQTITGTTQSAGAHTHKVTISPTGERETRPVNTTVVYAIKS

SEQ ID NO: 16 - protein, 324 aa, synthetic construct
MSNIYDWSLKADENAHSDNIINWSEGQPPSSVNDSARAMMQRVREYLADNGGSIDAGFMINAEDKTTLITLKTVSPIKKYNNDIIIRFKAHGVNVGKTTIKVNSMGEKPIYKATHTGVISLEGGELQTDGIYEMVYNGNISTEGRDGWYLLNPTSPKVESFPTGFIATFAMQEIPTGWLLCDGKAYKRKDYPQLFKAIGDKWGKDSDTTFKVPDFRGMFLRGFDDGRGLDDNRKFADEQQDSIKSHTHIGNIEESGEHTHNFQYKGVGWPTGNIGRLPNYYTYNATLQGTTDSAGAHTHKVTLSHTGETETRPVNTTVVYAIKS

SEQ ID NO: 17 - protein, 279 aa, synthetic construct
MNIKSLMTTSVIALISASAAQAADVIVPHEVAPTVISAPAFSWTGFYIGGQVGNFSSKVEITDPNKKDKLFSKDDTPKPSGFMGGIYAGSNMDLGNNMILGVETDAVWADREDAKTSSAEAIGQDELETFRDSLKKANAAFAQGKTSDNVAAVDKHTDSLALKEKWSGATRVRIGFTAADRIMPYVAGGVSYAQVQAVSSTKVTQAADDAEIATAQLFDKTKTLVGFTLGGGVDFAMTDNVLLRAEYRYSDFGKKKFEKEGSEFSYKTNDFRVGVAYKF

SEQ ID NO: 18 - protein, 272 aa, synthetic construct
MNIKSLITTSVIALVSASAAQAADVIATHEAAPVITTPTFSWTGFYIGGQIGNFTSDNKIKGLGKETSIFTKELTPQLSGIVGGIYAGSNIDLGSGLILGVETDAIWADCEATKTSAIRTLSVPQANSLNDEFKAAGITLKDKFSENDTMSDHYTYKAKWSGATRARIGFSAFDRVMPYFAGGVAYARMQGMKSVSGMNAAKNKKLGGGLYDETKMMVGFTVSGGVDVAMTGNVLLRGEYRYSDFGKKKFLNNTQEFNYKTNDFRFGVAYKF

SEQ ID NO: 19 - protein, 276 aa, synthetic construct
MNIKSLITTSVIALASASAVQAADVIIPHEAAPVISAPAFSWTGFYIGGQVGSFSSKVEITEPSKNKLNKDLTPKPSGFMGGVYAGSNIELGNNLILGVETDAVWADREETKERFSHVLEADELDAFKGDLTQLKASPATGKQIAAIKTGDKRTDSSTLKEKWSGATRVRIGFAAADHIMPYVAGGVAYAQMQGTYTIKATEAKGNAEIATAKLFDATKTFVGYTLGGGVDIAMTDNVLLRAEYRYSDYGKKQFDRDKDEFSYKTNDFRVGVAYKF

SEQ ID NO: 20 - protein, 294 aa, synthetic construct
MNIKFLMATSVVTLVSVSAANAADVIVPHEVAPAVVTAPSFSWTGFYLGGQIGNFSSKTKVTIPGEDKELFKKDNTPSPSGFMGGIYAGSNVDLGSGLILGVETDAVWADREESKTARESTLTKKDATFFNDALKKAKVELTGQQKFIEDDQVTETHSYKEKWSGATRVRIGFAAVDRIMPYVAGGIAYAQVQGIQSVAGKGERAKVEQKADGGEELPEGNKQTIDLTGGTWADDTKTMVGFTIGGGVDFAMTDNVLLRAEYRYSDFGKKKFANDTREFNYKTNDFRVGVAYKF

SEQ ID NO: 21 - protein, 288 aa, synthetic construct
MNIKCLVTASVCALISASAAQAADVMIPQEISPIISAPTFSWTGLYLGGQIGGLSGKHDFKAIGKDSEWPFANKDLKVSGFVGGLYAGSNIDLGSGLVLGVDTDIVWVDKEGKLSSNHKAETQDDADAFKQIFDENKIEVAKGQIKELTQNFSLKEKWAGATRVRIGFGADRIMPYVSGGVAYTQVQAIGSAILKGTKDTGTEGGGGSASKAVRSEALDVLASGTITDEKKTLLGYTLGAGVDFAMTDNVILRTEYRYSDFGKKKFVKDAIETNYKTNDFRVGVAYKF

SEQ ID NO: 22 - protein, 292 aa, synthetic construct
MNMKCLMTASVVALMTAYAAQAADITVPQETGETIVVAPAFSWTGFYVGGQIGGFSSKTSFSSRDGDDSKKWLPLHKDLWSKPSGFTGGFYTGSNVDLGNGLILGVDTDVIWSGRKDEKTHEVTVELEDKDKNKHQEKSVSRSPVVFADGEEKEVEITKYGHTLKEKWAGATRIRIGFAADRIMPYVAGGVAYTQLQDSFSTSVSRQKVSGAKAERADKEPSAEPNTVNTVDESKTMIGYTLGAGVDFAMTDNVVLRAEYRYSDFGKKKLHNDKLEVNYKANDFRVGVAYKF

SEQ ID NO: 23 - protein, 278 aa, synthetic construct
MNIKSLVTTSVIAMAAASAAQAADVIIPRETAPAVISAAPSFSWTGFYIGGQVGNFSGKVDEFDSETKKKITDKDWTPKPSGFMGGIYVGSNVDLGNGLILGVETDAVWADREDSKTHSAVIGETGLAAFKNKLTAAEATFASGKNIDNVTKDGKRVEGTSIKEKWSGATRVRIGFAAVDRILPYFAGGIAYTQLQVVNSLKAEGADGAEIASAKMFDQSKTMVGFTVGGGVDFAMTDNVLLRAEYRYSDFGKKAFVKDEDKIAYKTNDFRVGVAYKF

SEQ ID NO: 24 - protein, 279 aa, synthetic construct
MNIKSLMTTSVIALLSASAVQAADVIVPHEIAPAVITAPTFSWTGFYIGGQVGNFSSKVEVTDPNTKNKLFSKDDAPKPSGFMGGVYAGSNIDLGNNVILGVETDAVWADREDSKTLSSKAIEETKLGEFKASLTKANAVVATGKKIDDIKKEDKHTDSLVLKEKWSGATRVRIGFAAADRIMPYVSGGVSYVQVQAVSSTKVTQAADGAEIVSAKFFDKTKTLVGFTLGGGVDFAMTDNVLLRAEYRYSDFGKKKFEKDGSELSYKTNDFRVGVAYKF

SEQ ID NO: 25 - protein, 262 aa, synthetic construct
MNIKHLLVACSWTFIPVSMVQAADIMKPHQSQSYQPVPVISSSNNFSWTGFYVGAQIGGFSGEATLSPIQDSVEEDREGQGFSEIKSPKLSGFIGGIYAGSNFSLSKDLILGIDTDMVWSGKKNTKVFDVQDMSIDMSIKEKWAGAMRMRVGFAMDRVMPYVSGGVSYVQLQNVFTAKRLAKAVVRERRNTLPSDSEPKFLDETKILVGYSVGGGVDVAMTDNVIMRAEYRYSDFGKKKFAKDNVDLSYKTNDFRVGVAYKF

SEQ ID NO: 26 - protein, 208 aa, synthetic construct
TKDETPQPSGFMGGIYAGSNVDLGNGLILGVETDAVWADRGETKTISVFSGNSSETSNAGASQTPNAGAEEKKSISLKTTALVDAKIKNNFTFKEKWSGATRVRVGFAAADRILPYVAGGIAYAQMQGIATVSGKDASGKTVSTKESDKTMTMVGFTIGGGVDFAMTDNVLLRAEYRYSDFGKKKFEKDDAEFHYKTNDFRVGVAYKF

SEQ ID NO: 27 - protein, 245 aa, synthetic construct
MKKVIFKPLKSYPVKMAMIALTLLAASPITHAEESKMKNATITVTATGENQATPDMAIINLAVVTQDKTAQKALADNNKSMNDIINAFKNNGIQANDLQTSGLSIYQSNPNKDHEKKNNGIVYHVSNSLTVRIRDLSNAGKIFDQAMALGVNSVHGITFTNANTKPFYQEARKKAIAEAIEKAKTIAEAADVKLGKIIKINENDDNYYSRPHLMSKAVNANYTDTTFSSGELNYSVSVTIVFAID

SEQ ID NO: 28 - protein, 245 aa, synthetic construct
MTKIISQPLKSYSIKMAMITLTLLAASLPTHAEESKIKNATITVTATGESQATPDMAIINLAIVTHDKTAQKALAANNKSMNDIVNAFKNNGIQANDLQTSGLSIYQSSPEKHREKKNTEILYQVSNSLTVRIRDLSNAGKIFDQAMALGVNSVNGITFTNANTKPFYQEARKKAVTAAIEKAETIAQAANLKLGKIITINENDDNSYSRPHLMSRAAHASYADTTFSSGELNYNVSVTVVFAID

SEQ ID NO: 29 - protein, 245 aa, synthetic construct
MKKTTFQPLNHYSVRIAMVALALITSSLPIHAEESKMKNATITVTATGESQAAPDMAIINLAVVTEDKTAQKALADNNKSMNDIVNTFKDNGIQANDLQTSGLSIYQSSPDKPHDKKNHEKLYHVSNSLTVRIRDLSNAGKIFDQAMALGVNSVNGITFTNADTKPLYQEARKKAITEAIEKAETIAQAANLKLGKIIEINENNDNYYPKPRLMSSAAHASYADTNFSGGELGYNVSVTVVFAID

SEQ ID NO: 30 - protein, 238 aa, synthetic construct
IQPLKSSLVKIAILTFTLLTTSLAVVHAYAEESKRDATITVTAIGESQATPDMAIINLAVVTHDKTAQKALASNNQSINEIIKAFKNNGIQANDLQTSGLSIYQSSHEKKNNEKLYQVSNSLTVRIRDLSNAGKIFDQAMALGINSVNGITFTNADTKPFYQEARKKAITEAIEKAQTIAQAADLKLGKIIEINEKDDYYRPTPRLMSRAADASYADTNFAGGELNYNVSVTVVFAID

SEQ ID NO: 31 - protein, 246 aa, synthetic construct
MTKAISQLFNIYWTKITILTILALLTSSLSVYAEKSTIKNSTITVTATGESQATPDMAIMNLAVITHNKTAQKALEANNKSINNIIDSFKKEGIQEKDIQTSNLFIHYINSDKHQEQRGDENLYQVSHSLTVRIRDLLNAGMILDQAMALGINSVRDITFTNTDTKPFYTAARKQAIAEAIEKAETLAHAASVKLGKIIEINESNDPPHLMPRLISRAQTASYADTHFSSGELNYSVTVTVTFAID

SEQ ID NO: 32 - protein, 247 aa, synthetic construct
MTKIIFQLLNTYRIKLSILTVLSLFATSLFINAEETIKDAPTITVTATGENQATPDMAIINLAVVTHDTTAQKALAANNKFMNDIVNTFKNNGIQANDLQTSGLSIYQTNSDKKREKKNNEIIYHVSNSLTVNIRDLANAGKIFDQAMALGVNSVNGITFTNADTKPLYKEARKKAIAEAIEKAETLAQAANVKLGKIITISENNNGHYPTPRLMSRAQHARSYTDTNFSGGELNYNVSVTITFAID

SEQ ID NO: 33 - protein, 231 aa, synthetic construct
VILAFTLLATSLTVRAYAEENKMKNATITVTAIGESQAAPDMAIINLAVVTYDKTAQKALASNNQSINDIIKAFKNDGIQANDLQTSGLSIYQSTSDKHHEKKNNEKLYHVSNSLTVRIRDLNNAGKIFDQAMTLGVNSVNGITFTNADTKPFYQEARKKAITEAIEKAQTIAQAADLKLGKIIEINERDDYYHPTPRLMSRAADASYENTNFAGGELTYNVSVTVVFAID

SEQ ID NO: 34 - protein, 177 aa, synthetic construct
QKALADNNKSMNDIVNAFKNNGIQENDLQTSGLSIYQSNQGKHHEKKNNGNGIVYHVSNSLTVRIRDLSNAGKIFDQAMALGVNSVHGITFTNADTKPFYQEARKQAITEAIEKAKTIAEAANLKLGKIIKINETDDNYYSRPHLMSKTVNASYTDTAFSSGELNYNVSVTVVFAIN

SEQ ID NO: 35 - protein, 246 aa, synthetic construct
MTKMIFQLLKVYRIKLSILTVLSLLTTSCFVHAEETIKNAPTITVTATGENQATPDMAIINLAVVTHDTTAQKALAANNKFMNDILDTFKNNGIQANDLQTSGLSIYQTNSDKKQEKKNNEITYHVSNSLTVNIRNLANAGKIFDQAMALGVNSVNGITFTNADTKPLYKEARKKAVIEAIAKAETLAQAANVKLGKIITINESNNEYYPTPRLMSRAQSASYADTNFSGGELNYHVSVTITFAID

SEQ ID NO: 36 - protein, 175 aa, synthetic construct
QEALAANNKSMNDIVNAFKSNGIQANDLQTSNLSIYQSSPDNHHEKKNKEILYRVSNSLTVRIRDLANAGKIFDQAMALGINSVNGITFTNADMKPFYQDARKKAIAEAIEKAETIAQAAHLKLGKIIEINENSDGYHPSPRLMSRAANASYADTNFSGGELNYSVSVTVVFTID

SEQ ID NO: 37 - protein, 148 aa, synthetic construct
MKKYSLVTLLSLFCISHAKAQTAPLTDEYYKKALENTQKLDVAKSQTAQSIYESATQTANKIKDINNQLANLKADTKTKPEQLQALQIELSLLQAQLQADTLKIQSLAMIQAKDTKTKEELREEQTQKKHEDLQKQLKEKLEKSDVRL

SEQ ID NO: 38 - protein, 160 aa, synthetic construct
MAAYISPKERKSMKKYGLVTLLSLSCVSHTIAETALLADEYYKRALENTQKLDAAKSETAESIYASANEITNKIKEINEKLRKAQAAEKTKPEEFQALQIELSLLQAQLQADTLKIQFLSMIQAKNTKTKEDIREEQTQKKHKDLQEKLKEKLGNSDVRL

SEQ ID NO: 39 - protein, 155 aa, synthetic construct
MKKYSLVTLLSSFFISHAVANNSKDVDEYYKQALESTQKLNVVKSETAETIFDHATQTAKKIQEISEQLKQKQSQEKPDAEKLQTLQVELSVLQAKLQAEALKLQSLDMIQAKDSRTKEEIREEEIKKQHEKIVQKLKEKLEKSNVPSTLGKLND

SEQ ID NO: 40 - protein, 177 aa, synthetic construct
MKKYGLMTLLSFSFISHAFSQMSPDADEYYKQALENTQKLDTAKSETAETIYKTATETAKKIKEINGQLEQLKSQTETTATETTSDADKNSNLEELQKRQALRQELQVKVSLLQADLQANSLKLQALNMIQARDTKTKEELREEKEQQKHKTLQAQLKEKEEKLKEKLESTGADVRL

SEQ ID NO: 41 - protein, 148 aa, synthetic construct
MKKYSLVTLLSLFCISHAKAQIAPLPDEYYKKALENTQKLDVAKSQTAESIYESATQTANKIKDINDQLKTLKADTKTKPEQLQALQIELTLLQAQLQADTLKIQSLSMIQAKDTKTKEELREEQTQQNHKKIEEKLKEKLEKSDVRL

(19) United States
(12) Patent Application Publication
(10) Pub. No.: US 2024/0192209 A1
(43) Pub. Date: Jun. 13, 2024

(54) DIAGNOSIS OF BARTONELLA USING RECOMBINANT PROTEINS

(71) Applicant: ID-Fish Technology, Inc., Milpitas, CA (US)

(72) Inventors: Jyotsna S. Shah, Santa Clara, CA (US); Song Liu, San Jose, CA (US); Prerna Bhargava, Sunnyvale, CA (US); Hari-Hara Potula, San Jose, CA (US); Iris Cruz, Freemont, CA (US)

(73) Assignee: ID-Fish Technology, Inc., Milpitas, CA (US)

(21) Appl. No.: 18/533,661

(22) Filed: Dec. 8, 2023

Related U.S. Application Data
(60) Provisional application No. 63/386,990, filed on Dec. 12, 2022.

(51) Int. Cl. G01N 33/569

(52) U.S. Cl. CPC G01N 33/56911; G01N 2469/20

(57) ABSTRACT

The disclosure, in some aspects, provides antigen-specific amino acid sequences for Bartonella species.

### RELATED APPLICATIONS

This application claims benefit under 35 U.S.C. § 119(e) of U.S. provisional application Ser. No. 63/386,990, filed Dec. 12, 2022, the disclosure of which is incorporated by reference herein in its entirety.

### FIELD

Aspects of the present disclosure provide diagnostic panels and methods for diagnosing infection by Bartonella species.

### BACKGROUND

Bartonellosis is a group of emerging infectious diseases caused by bacteria belonging to the Bartonella genus. Bartonella includes at least 22 named species of bacteria that are mainly transmitted by carriers (i.e., vectors), including fleas, keds, lice, sandflies, ticks and, potentially, mites and spiders. Both domestic and wild animals can be infected with Bartonella species (i.e., Bartonella spp) by these vectors. Among the Bartonella spp, at least 14 have been implicated in diseases that can be transmitted from animals to people (i.e., zoonotic disease). Of these zoonotic species, several may be transmitted to humans by companion animals (e.g., dogs and cats), typically through a bite or scratch.

Human diseases that have been identified as being caused by one of the Bartonella spp bacteria include cat scratch disease caused by Bartonella henselae, Carrion's disease caused by Bartonella bacilliformis, and trench fever caused by Bartonella quintana. Bartonella spp have also been associated with diseases of the skin (i.e., bacillary angiomatosis), liver (i.e., peliosis hepatis), heart (i.e., endocarditis), eyes (i.e., neuroretinis), blood (i.e., bacteremia), and brain (i.e., encephalitis).

Bartonella infection does not always cause overt illness. A number of studies have detected clinically healthy people that have tested positive (i.e., seropositive) for Bartonella, but have no known history of typical Bartonella symptoms. Those who do become ill usually develop mild disease that tend to end without treatment (i.e., are self-limiting).

Bartonella was thought to cause severe infection in some people. Immunocompromised patients, such as those undergoing immunosuppressive treatments for cancer, organ transplant patients, and people with human immunodeficiency virus (HIV)/acquired immunodeficiency syndrome (AIDS), are more likely to develop severe, life-threatening disease. Recent advances in diagnostic techniques have facilitated documentation of chronic bloodstream and dermatological infections with Bartonella spp. in human blood donors, and in immunocompetent and immunocompromised human patients.

Prior to 1990, there was only one named Bartonella species (i.e., B. bacilliformis), whereas there are now over 36, of which 17 have been associated with an expanding spectrum of animal and human diseases.

The field of Bartonella research remains in its infancy and is rich in questions, for which patient relevant answers are badly needed. Directed Bartonella research could substantially reduce a spectrum of chronic and debilitating animal and human diseases, and thereby reduce suffering throughout the world.

### REFERENCE TO AN ELECTRONIC SEQUENCE LISTING

The contents of the electronic sequence listing (SEQlisting.xml; Size: 47.4 KB; Date of Creation: Dec. 7, 2023) is herein incorporated by reference in its entirety.

### SUMMARY

The present disclosure provides diagnostic panels and methods for diagnosing, and treating, Bartonellosis resulting from infection by diverse Bartonella species.

A first aspect of the present disclosure relates to a diagnostic panel comprising individually identifiable labelled, tagged, or bound amino acid sequences comprising SEQ ID NOs: 1-41, and variants thereof that retain the immunological binding profile of the corresponding non-variant. In some embodiments of the first aspect, the amino acid sequences are bound to one or more substrates selected from the group consisting of nitrocellulose, nylon, polyvinylidene difluoride (PVDF), magnetic beads, agarose, plastic, and metal. In some embodiments of the first aspect, the amino acid sequences comprise one or more labels selected from the group consisting of stable isotopes, mass tags, and fluorophores. In some embodiments of the first aspect, the amino acid sequences comprise one or more peptide sequence tags selected from the group consisting of affinity tags, chromatography tags, epitope tags, enzyme conjugate tags, fluorescence tags, and combinations thereof.

A second aspect of the present disclosure relates to a method for detecting infection by one or more Bartonella species, the method comprising: contacting a biological sample, obtained from a subject suspected of having a Bartonella infection, with a diagnostic panel comprising amino acid sequences comprising SEQ ID NOs: 1-41, or one or more variants thereof that retain the immunological binding profile of the corresponding non-variant, wherein the biological sample contains at least one of IgM-, IgG-, and IgA-class antibodies; and determining the biological sample is positive for Bartonella infection when: a positive immunobinding reaction with IgM-, IgG-, or IgA-class antibodies is detected for at least one of SEQ ID NOs: 1-12 and at least one of SEQ ID NOs: 13-41, or a positive immunobinding reaction with IgM-, IgG-, or IgA-class antibodies is detected for at least one of SEQ ID NOs: 1-4, at least one of SEQ ID NOs: 5-8, and at least one of SEQ ID NOs: 9-12. In some embodiments of the second aspect, the amino acid sequences are unlabelled, untagged, and unbound. In some embodiments of the second aspect, the amino acid sequences comprise one or more labels selected from the group consisting of stable isotopes, mass tags, and fluorophores. In some embodiments of the second aspect, the amino acid sequences comprise one or more peptide sequence tags selected from the group consisting of affinity tags, chromatography tags, epitope tags, enzyme conjugate tags, fluorescence tags, and combinations thereof. In some embodiments of the second aspect, the amino acid sequences are bound to one or more substrates. In some embodiments of the second aspect, the one or more substrates is selected from the group consisting of nitrocellulose, nylon, polyvinylidene difluoride (PVDF), magnetic beads, agarose, plastic, and metal. In some embodiments of the second aspect, the immunobinding of IgM-class antibodies is detected through the use of an anti-human IgM antibody linked to a detectable moiety. In some embodiments of the second aspect, the immunobinding of IgG-class antibodies is detected through the use of an anti-human IgG antibody linked to a detectable moiety. In some embodiments of the second aspect, the immunobinding of IgA-class antibodies is detected through the use of an anti-human IgA antibody linked to a detectable moiety. In some embodiments of the second aspect, the detectable moiety is selected from the group consisting of chromophores, radioactive moieties, and enzymes. In some embodiments of the second aspect, the detectable moiety comprises alkaline phosphatase. In some embodiments of the second aspect, the detectable moiety comprises biotin. In some embodiments of the second aspect, the method comprises, in response to determining the biological sample is positive for the Bartonella infection, treating the subject for the Bartonella infection. In some embodiments of the second aspect, the method comprises: determining the biological sample is positive for Bartonella henselae infection when a positive immunobinding reaction with IgM-, IgG-, or IgA-class antibodies is detected for at least one of SEQ ID NOs: 13, 17, and 27; determining the biological sample is positive for Bartonella quintana infection when a positive immunobinding reaction with IgM-, IgG-, or IgA-class antibodies is detected for at least one of SEQ ID NOs: 14, 18, and 28; determining the biological sample is positive for Bartonella vinsonii infection when a positive immunobinding reaction with IgM-, IgG-, or IgA-class antibodies is detected for at least one of SEQ ID NOs: 15, 19, and 29; and determining the biological sample is positive for Bartonella elizabethae infection when a positive immunobinding reaction with IgM-, IgG-, or IgA-class antibodies is detected for at least one of SEQ ID NOs: 16, 20, and 30. In some embodiments of the second aspect, the method comprises, in response to determining the biological sample is positive for the Bartonella henselae infection, treating the subject for the Bartonella henselae infection. In some embodiments of the second aspect, the method comprises, in response to determining the biological sample is positive for the Bartonella quintana infection, treating the subject for the Bartonella quintana infection. In some embodiments of the second aspect, the method comprises, in response to determining the biological sample is positive for the Bartonella vinsonii infection, treating the subject for the Bartonella vinsonii infection. In some embodiments of the second aspect, the method comprises, in response to determining the biological sample is positive for the Bartonella elizabethae infection, treating the subject for the Bartonella elizabethae infection.

A third aspect of the present disclosure relates to a diagnostic panel comprising individually identifiable labelled, tagged, or bound amino acid sequences comprising 1, 2, 3, or 4 of SEQ ID NOs: 1-4; 1, 2, 3, or 4 of SEQ ID NOs: 5-8, and 1, 2, 3, or 4 of SEQ ID NOs: 9-12, and variants thereof that retain the immunological binding profile of the corresponding non-variant. In some embodiments of the third aspect, the diagnostic panel comprises individually identifiable labelled, tagged, or bound amino acid sequences comprising SEQ ID NOs: 1-12, and variants thereof that retain the immunological binding profile of the corresponding non-variant. In some embodiments of the third aspect, the amino acid sequences are bound to one or more substrates selected from the group consisting of nitrocellulose, nylon, polyvinylidene difluoride (PVDF), magnetic beads, agarose, plastic, and metal. In some embodiments of the third aspect, the amino acid sequences comprise one or more labels selected from the group consisting of stable isotopes, mass tags, and fluorophores. In some embodiments of the third aspect, the amino acid sequences comprise one or more peptide sequence tags selected from the group consisting of affinity tags, chromatography tags, epitope tags, enzyme conjugate tags, fluorescence tags, and combinations thereof.

A fourth aspect of the present disclosure relates to a method for detecting infection by one or more Bartonella species, the method comprising: contacting a biological sample, obtained from a subject suspected of having a Bartonella infection, with a diagnostic panel comprising individually identifiable labelled, tagged, or bound amino acid sequences comprising 1, 2, 3, or 4 of SEQ ID NOs: 1-4; 1, 2, 3, or 4 of SEQ ID NOs: 5-8, and 1, 2, 3, or 4 of SEQ ID NOs: 9-12, and variants thereof that retain the immunological binding profile of the corresponding non-variant. In some embodiments of the fourth aspect, the diagnostic panel comprises amino acid sequences comprising SEQ ID NOs: 1-12, or one or more variants thereof that retain the immunological binding profile of the corresponding non-variant, wherein the biological sample contains at least one of IgM-, IgG-, and IgA-class antibodies; and determining the biological sample is positive for Bartonella infection when a positive immunobinding reaction with IgM-, IgG-, or IgA-class antibodies is detected for at least one of SEQ ID NOs: 1-4, at least one of SEQ ID NOs: 5-8, and at least one of SEQ ID NOs: 9-12. In some embodiments of the fourth aspect, the amino acid sequences are unlabelled, untagged, and unbound. In some embodiments of the fourth aspect, the amino acid sequences comprise one or more labels selected from the group consisting of stable isotopes, mass tags, and fluorophores. In some embodiments of the fourth aspect, the amino acid sequences comprise one or more peptide sequence tags selected from the group consisting of affinity tags, chromatography tags, epitope tags, enzyme conjugate tags, fluorescence tags, and combinations thereof. In some embodiments of the fourth aspect, the amino acid sequences are bound to one or more substrates. In some embodiments of the fourth aspect, the one or more substrates is selected from the group consisting of nitrocellulose, nylon, polyvinylidene difluoride (PVDF), magnetic beads, agarose, plastic, and metal. In some embodiments of the fourth aspect, the immunobinding of IgM-class antibodies is detected through the use of an anti-human IgM antibody linked to a detectable moiety. In some embodiments of the fourth aspect, the immunobinding of IgG-class antibodies is detected through the use of an anti-human IgG antibody linked to a detectable moiety. In some embodiments of the fourth aspect, the immunobinding of IgA-class antibodies is detected through the use of an anti-human IgA antibody linked to a detectable moiety. In some embodiments of the fourth aspect, the detectable moiety is selected from the group consisting of chromophores, radioactive moieties, and enzymes. In some embodiments of the fourth aspect, the detectable moiety comprises alkaline phosphatase. In some embodiments of the fourth aspect, the detectable moiety comprises biotin. In some embodiments of the fourth aspect, the method comprises, in response to determining the biological sample is positive for the Bartonella infection, treating the subject for the Bartonella infection.

A fifth aspect of the present disclosure relates to a diagnostic panel comprising individually identifiable labelled, tagged, or bound amino acid sequences comprising SEQ ID NOs: 13-20 and 27-30, and variants thereof that retain the immunological binding profile of the corresponding non-variant. In some embodiments of the fifth aspect, the amino acid sequences are bound to one or more substrates selected from the group consisting of nitrocellulose, nylon, polyvinylidene difluoride (PVDF), magnetic beads, agarose, plastic, and metal. In some embodiments of the fifth aspect, the amino acid sequences comprise one or more labels selected from the group consisting of stable isotopes, mass tags, and fluorophores. In some embodiments of the fifth aspect, the amino acid sequences comprise one or more peptide sequence tags selected from the group consisting of affinity tags, chromatography tags, epitope tags, enzyme conjugate tags, fluorescence tags, and combinations thereof.

A sixth aspect of the present disclosure relates to a method for detecting species-specific infection by Bartonella henselae, Bartonella quintana, Bartonella vinsonii, and/or Bartonella elizabethae, the method comprising: contacting a biological sample, obtained from a subject determined to have a Bartonella infection, with a diagnostic panel comprising amino acid sequences comprising SEQ ID NOs: 13-20 and 27-30, or one or more variants thereof that retain the immunological binding profile of the corresponding non-variant, wherein the biological sample contains at least one of IgM-, IgG-, and IgA-class antibodies; determining the biological sample is positive for Bartonella henselae infection when a positive immunobinding reaction with IgM-, IgG-, or IgA-class antibodies is detected for at least one of SEQ ID NOs: 13, 17, and 27; determining the biological sample is positive for Bartonella quintana infection when a positive immunobinding reaction with IgM-, IgG-, or IgA-class antibodies is detected for at least one of SEQ ID NOs: 14, 18, and 28; determining the biological sample is positive for Bartonella vinsonii infection when a positive immunobinding reaction with IgM-, IgG-, or IgA-class antibodies is detected for at least one of SEQ ID NOs: 15, 19, and 29; and determining the biological sample is positive for Bartonella elizabethae infection when a positive immunobinding reaction with IgM-, IgG-, or IgA-class antibodies is detected for at least one of SEQ ID NOs. 16, 20, and 30. In some embodiments of the sixth aspect, the amino acid sequences are unlabelled, untagged, and unbound. In some embodiments of the sixth aspect, the amino acid sequences comprise one or more labels selected from the group consisting of stable isotopes, mass tags, and fluorophores. In some embodiments of the sixth aspect, the amino acid sequences comprise one or more peptide sequence tags selected from the group consisting of affinity tags, chromatography tags, epitope tags, enzyme conjugate tags, fluorescence tags, and combinations thereof. In some embodiments of the sixth aspect, the amino acid sequences are bound to one or more substrates. In some embodiments of the sixth aspect, the one or more substrates is selected from the group consisting of nitrocellulose, nylon, polyvinylidene difluoride (PVDF), magnetic beads, agarose, plastic, and metal. In some embodiments of the sixth aspect, the immunobinding of IgM-class antibodies is detected through the use of an anti-human IgM antibody linked to a detectable moiety. In some embodiments of the sixth aspect the immunobinding of IgG-class antibodies is detected through the use of an anti-human IgG antibody linked to a detectable moiety. In some embodiments of the sixth aspect, the immunobinding of IgA-class antibodies is detected through the use of an anti-human IgA antibody linked to a detectable moiety. In some embodiments of the sixth aspect, the detectable moiety is selected from the group consisting of chromophores, radioactive moieties, and enzymes. In some embodiments of the sixth aspect, the detectable moiety comprises alkaline phosphatase. In some embodiments of the sixth aspect, the detectable moiety comprises biotin. In some embodiments of the sixth aspect, the method comprises, in response to determining the biological sample is positive for the Bartonella henselae infection, treating the subject for the Bartonella henselae infection. In some embodiments of the sixth aspect, the method comprises, in response to determining the biological sample is positive for the Bartonella quintana infection, treating the subject for the Bartonella quintana infection. In some embodiments of the sixth aspect, the method comprises, in response to determining the biological sample is positive for the Bartonella vinsonii infection, treating the subject for the Bartonella vinsonii infection. In some embodiments of the sixth aspect, the method comprises, in response to determining the biological sample is positive for the Bartonella elizahethae infection, treating the subject for the Bartonella elizaethae infection.

### BRIEF DESCRIPTION OF THE DRAWINGS

FIG. 1 presents images showing results of IgM- and IgG-class Bartonella ImmunoBlots used to test nine human samples suspected of Bartonella infection.

FIG. 2A-B presents comparison images of Bartonella Western Blots (FIG. 2A) with Bartonella ImmunoBlots (FIG. 2B) of the present disclosure.

DESCRIPTIQN QF SEQUENCES

SEQ ID NO: 1-amino acid sequence of a Bartonella henselae htRA recombinant protein
MVKKTFFTTLAAVSFSAALETALFFSGCGSSLWTTKAHANSVFSSLMQQQGFADIVSQV
KPAVVSVQVKSNKKKKEWFFSDFFSTPGFDQLPDQHPLKKFFQDFYNRDKPSNKSLQRS
HRLRPIAFGSGFFISSDGYIVTNNHVISDGTSYAVVLDDGTELNAKLIGTDPRTDLAVLKV
NEKRKFSYVDFGDDSKLRVGDWVVAIGNPFGLGGTVTAGIVSARGRDIGTGVYDDFIQI
DAAVNRGNSGGPTFDLNGKVVGVNTAIFSPSGGNVGIAFAIPAATAKQVVQQLIEKGLV
QRGWLGVQIQPVTKEISDSIGLKEAKGALITDPLKGPAAKAGIKAGDVIISVNGEKINDV
RDLAKRIANMSPGETVTLGVWKSGKEENIKVKLDSMPEDENMKDGSKYSNEHGNSDET
LEDYGLIVAPSDDGVGLVVTDVDPDSDAADKGIRPGDVIVTVNNKSVKKVSDITDTIKN
AQKLGRKAILLQVRTNDQNRFVALPIFKK
SEQ ID NO: 2-amino acid sequence of a Bartonella quintana htRA recombinant protein
MVKKTFFPTLAAVGFSAALETALFFSGCGSSLWMTEARANSVFASLVQQQGFANIVSQV
KPAVVSVQVKSNKKKKEWFFSDFFSSPGLDQLPDQHPLKKFFQEFYDRGKPSNKSLKRP
HRLRPIAFGSGFFISSDGYLVTNNHVISDGTSYTVVLDDGTELNAKLIGTDARTDLAVLK
VDEKRTFSYVDFGDDSKLRIGDWVVAIGNPFGLGGTVTAGIVSARGRDIGTGVYDDFIQI
DAAVNRGNSGGPTFDLNGKVIGVNTAIFSLSGGNVGIAFAIPAAIAKQVVQQLIEKGSVQ
RGWLGVQIQPVTKEISDSIGLKEAKGALITDPLKGPAAKAGIKAGDVIISVNGEKINDVR
DLAKRIANIKPGETVTLGVWKSGKEENIKVKLDTMPDDKVMKEDSKYSNGRNDSNETL
EDYGLIVAPSDDGIGLVVTDVDSDSDAADKGIRPGDVIVTVNNKSVKKVSDITDTIKNAQ
KLGRKAILLQVRINDQNRFVALPIFKK
SEQ ID NO: 3-amino acid sequence of a Bartonella vinsonii htRA recombinant protein
MVKKTFFKTLIAVSFSAALESALFFSGCGSSLWTTAAHANSVFTSFMQQPGFADIVSQVK
PAVVSVQVKSNKKKDEWSFSNFFSGPGFDQLPDQHPLKRFFKEFYDLDKPKNKFPSHSH
RLRPIAFGSGFFISSDGYIVTNKHVISDGTSYSVVLDDGTELNAKLIGSDQRTDLAVLKVN
DKRKFSYVDFGDDSKLRVGDWVVAIGNPFGLGGTVTAGIVSARGRDIGTGVYDDFIQID
AAVNRGNSGGPTFDLNGKVVGVNTAIFSPSGGNVGIAFAIPAATVKQVVQQLIEKGSVQ
RGWLGVQIQPVTKEISDSIGLKEDKGALVTDPLKGPAEKAGIKAGDVIISVNGEKINDAR
DLAKRIANIRPGETVTLGVWRSGKEENIKVKLASMPEDEGKKEGSKYSNKRNDADETLE
DYGLIVAPSDDDVGLVVTDVDSDSDAADKGIRPGDVIITVNNKSVKKVSDITDTIKNAQ
KLGRKAILLQVRINGQNRFVALPIFKK
SEQ ID NO: 4-amino acid sequence of a Bartonella elizabethae htRA recombinant protein
MLKKTFFKTFVAVSFSAVLESALFFSGCTASLWTTKAHASSVFTSLVQQQGFADIVAQV
KPAVVAVQVKSNKKKEDWFFSNFFSGPGIDQLPDQHPLKRLFKEFYDFDKPKNKFPHHS
QRLRPIAFGSGFFISSDGYIVINDHVISEGTSYSVVLDDGTELNAKLIGKDPKTDLAVLKV
NDKRKFSYVDFGDDSKLRVGDWVVAIGNPFGLGGTVTAGIVSARGRDIGTSSYDDFIQI
DAAVNRGNSGGPTFDLNGKVVGVNTAIFSPSGGNVGIAFAIPAGTAKQVVQQLIEKGSV
QRGWLGVMIQPVTKEISDSIGLQEAKGALVTDPLKGPAAKAGIKAGDVIISVNDEKVND
SRDLAKRIANMSPQETVTLGIFRSGKEEKIKVKLAAMPEDEGKKESSKYLNERGNSDET
LEDYGLIVAPSEDGLGLVVTDVDSDSDAADKGIRPGDVIVTVNNKSVKKTSDITDAIKN
AQKLGRKAILLQVRTNDQNRFVALPIFKK
SEQ ID NO: 5-amino acid sequence of a Bartonella henselae SucB recombinant protein
MTTEIRVPTLGESVTEATVGKWFKKLGEAVAVDEPLIELETDKVTVEVPSPVAGKLSEII
AKEGDTVEVKALLGLVEAGAAGISQSFSPSATPIPEVPSELKQSSSSGAMQKDTMPPSPS
AAKLMAENNIAKSNISGSGKRGQILKEDVLGVLEQEVKAPSVSAASSSASLVQEKHEER
VRMTKLRQTIARRLKDAQNTAAMLTTFNEVDMSAVMDLRKRYKDLFEKKHGVKLGF
MGFFTKAVCHALKELPAVNAEIDGTDIVYKNYVNVGIAVGTDKGLVVPVVRHADQMS
LAEIEKEIGRLGRLARDGKLAVSDMQGGTFTITNGGVYGSLMSTPILNAPQSGILGMHAI
KERAMVVGGQIVIRPMMYLALSYDHRIVDGQEAVTFLVRVKESLEDPERLVLDL
SEQ ID NO: 6-amino acid sequence of a Bartonella quintana SucB recombinant protein
MTTGIRVPTLGESVTEATIGKWFKKLGEAVAVDEPLVELETDKVTVEVPSPVMGKLTEII
AKEGDIVEVNAVLGFVESGAAGISQSFSPSATSIPEAPSELEQSPSSSATPSGTMPPAPSAA
KLMAENNIAKSDISGSGKRGQILKEDVLGALAQGTKASTSVATLTASSSSAAPIQEMREE
RVRMTKLRQTIARRLKDAQNTAAMLTTFNEVDMSAVMDLRKRYKDLFEKKHGVKLGF
MGFFTKAVCHALKEFPTVNAEIDGTDIVYKNYVNAGIAVGTDKGLVVPVVRDADQMSL
AEIEKEISRLGRLARDGKLAVSDMQGGTFTITNGGVYGSLMSTPILNAPQSGILGMHAIK
ERAMVVGGQIIICPMMYLALSYDHRIVDGQEAVTFLVRVKESLEDPERLVLDL
SEQ ID NO: 7-amino acid sequence of a Bartonella vinsonii SucB recombinant protein
MTTEIRVPTLGESVTEATVGKWFKKLGEAVAIDEPLVELETDKVTVEVPSPVAGKLFEII
AKEGDTVEVNALLGAVEAGAASVAKSPSSSETSVSAAPSELEQSSSSNTMPPAPSAAKL
MAENNIAKSDILGSGKRGQILKEDVLNVLAQGVKTSPPAVSASSSTPVSVSSSAVAPVQE
MREERVRMTKLRQTIARRLKDAQNTAAMLTTFNEVDMSAVMGLRKRYKDLFEKKHG
VKLGFMGFFTKAVCHALKELPAVNAEIDGTDIIYKNYVNAGIAVGTDKGLVVPVVRDA
DQMSLAEIEKEIGRLGRLARDGKLAVSDMQGGTFTITNGGVYGSLMSTPILNAPQSGILG
MHAIKERAMVVDGQIAIRPMMYLALSYDHRIVDGQEAVTFLVRVKESLEDPERLVLDL
SEQ ID NO: 8-amino acid sequence of a Bartonella elizabethae SucB recombinant protein
MTTEIRVPTLGESVTEATVGKWFKKLGEAVAMDEPLVELETDKVTVEVPSPVAGKLSEII
AKEGDTVEVNALLGTVEAGAAGVTQSFSPSATSVPVASSESEKLASSNTMPPSPSAAKL
MAENNIAKSDIAGSGKRGQILKEDVLGGLEQKTKTPTPSSSATGSSVVSVPETREERVRM
TKLRQTIARRLKDAQNVAAMLTTFNEVDMSAVMDLRKRYKDLFEKKHGVKLGFMGFF
TKAVCHALKELPAVNAEIDGTDILYKNYVNVGIAVGTDKGLVVPVVRDADQMSLAEIE
KEIGRLGRLARDGKLAVSDMQGGTFTITNGGVYGSLMSTPILNAPQSGILGMHAIKERA
MVVEGQVVIRPMMYLALSYDHRIVDGQEAVTFLVRVKESLEDPERLVLDL
SEQ ID NO: 9-amino acid sequence of a Bartonella henselae N1pD/LppB recombinant protein
MCLKVLGNVVRCNQGIALLAMIMVLVGCSSGMQRFANPFSYPTISNQPNISTTISTYTDS
PMLQYGAGMIQSTELPPVEPVNDLSTYDNTAYNSPPQDGTSSPNSRIMGTPPRNLGTLSR
SQMRNDPLFRQNSYIVQTGDTLLSIARQRGVSVEALKLVNGIRSNSIYIGQVLMIPSGRTA
ETSNVRNNDWGASKQSLSQSQSASSIRHKKYSSTEKAPITPKPSAQINRSNGEQNSSAQM
SSLNYEKGVLDTVMNKDNGTTPQATGISKMRWPVRGRLLSQFGQKKGTTSRGIDIAVPE
GSSVKAAENGIVIYASDGLKELGNVVMIRHENNIITIYGCNSKLVVTRGQRIRRGDEIAKS
GVSGDVKTPRVYFEVRENSLPVDPIKYLEN
SEQ ID NO: 10-amino acid sequence of a Bartonella quintana N1pD/LppB recombinant
protein
MYLKVLGKIFRCNQGITLLAMITILAGCSSGTQRFANIFSHHTVSNRSNMSTTIPTDPGML
SYGGGVIQSTELPPVEPSNDSWADDSAPYNSPRQDGTLSPDGRIIGTPPRNLGTISRSQMN
NTPLFRRNSYIVQSGDTLLSIARQIGISVEALKLANGISSNNSIYIGQVLMIPGRRTVATSN
AQNDDRVTSTEPSSQSQVRSSIRYQKAPSTDKAPITAMPSSKINRSNAEQDSSTQMPSSN
YKTDVLDTVMNTDDGVTPQATGISKMRWPVRGRLLSQFGQKKGTATSRGIDIAVPEGS
SVKAAENGIVIYASDGLKELGNVVMIRHENDIITIYGYNSKLVVNRGQRIRRGDEIAKSG
VSGDVKTPRVYFEVRKNSLPVDPIEYLEN
SEQ ID NO: 11-amino acid sequence of a Bartonella vinsonii N1pD/LppB recombinant
protein
MCLKVLSNIFWRNFQGIFFLAMVTIFAGCSSGTQRFANIFPHRTESTHSNISTTMSADPQM
LPYGGMIQSTELPPVESGSDSWINDNTAYNSTQPRGETSSDSRIMGTPPRDLGTLSRSQM
GNSPIFRRNSYIVQSGDTLLSIARQIGINVEALKSANGIRNNSIYIGQVLMIPNSRTLATSNT
TNDKRVVSTTETSVQSQNTSAIRYKKASPINKTSATISSSAKINRSNIEKNSSKQMMQLN
HEASLPDTITNTDSIVTPQATGISKMRWPVRGRLLSQFGQKKGTATNRGIDIAVPEGSSV
KAAENGVVIYASDGLKELGNVVMIRHEDNIITIYGCNSRLVVNKGQRIRRGDEIAKSGIS
GNVKTPRVYFEVRKNAVPVDPTEYLEN
SEQ ID NO: 12-amino acid sequence of a Bartonella elizabethae N1pD/LppB recombinant
protein
MCLKALDNIFRHNFQRITFLAVVTIVAGCSSGSQRFSNIVSHHRVSSQSNIANSMSSDPG
MLSYGGEMIQSTELPPVEPSDDSWIDDNSPQQDRGAPSNGRVMGAPPRNLGTLSRSQM
DSSPIFRQNSYIVQSGDTLLSIARQVGVSVEALKSANGINSNSIYIGQVLVLPSRRALATSK
VPSYNDLAVSKAESSFQSQMTSSTKHKKASPIYKAPVTTPPSTQVNRSNSEKNSSKQMM
QLNHESDLSSTVTRTDNIKTPQATGISKMRWPVRGRLLSQFGQKKGTIMNRGIDIAVPEG
SSVKAAENGVVIYAGDGLKELGNVVMIRHENNIITIYGCNSQLVVNKGQRIRRGDEIAKS
GVSGNVKTPRVYFEMRKNSLPVDPLKYLEN
SEQ ID NO: 13-amino acid sequence of a Bartonella henselae 35 kDa recombinant protein
MSNIYDWSLKADENAHSDNIINWAEGQPPSSVNDSARAMMQRVREYLADNGGSLDSTF
IVNAEDKTTSIIVTTASPIEEYKNDIIIRFKAGGVNVGTTTVIVNNIEEKLLYKATNAGVTP
LEGGELQRDAIYEMVYNKDVSMEDHGGWYLLNPTPLPPPKIETFPSGFIATFAMQEVPS
GWLLCDGAVYERKDYPQLFKAIGDKWGKDSDTTFKVPDFRGMFLRGFDDGRGLDAGR
QFADQQHDSIRSHTHIGTIEEAGEHTHKFQYYGVGWNSGDIGRRNPFYYRQSSIGVTQSA
GAHTHNISLSSTGEAETRPVNATVVYAIKS
SEQ ID NO: 14-amino acid sequence of a Bartonella quintana 35 kDa recombinant protein
MSSIYDWSLEAAENAYADVNINWAEGQPPSSVNNSARVMMQRIKEYLLDNGGVMETQ
FTINEDKTSIRLITNSPIEAYMDGIVVRFKAQKQNRGITNIALKQLPMQPVYKITKSGIAPL
KGQEIQIGGLYELVYICNIDGKNTDGWFLINPVDKDNFPSGFIGTFAMEKLPDGWLVCD
GKEYSRKEYADLFEALGETWGKGDGQTTFNVPDLRGMFLRGLDSGKGIDKKRSLGTKQ
EESFKAHTHEGTTERAGEHTHKYEVYTKSIDIIASTNGWEAMYNHPNKNKVWETRSAG
AHEHKISLKKTGGDETRPVNVAVVYAVKA
SEQ ID NO: 15-amino acid sequence of a Bartonella vinsonii 35 kDa recombinant protein
MSNIYDWSLKADENANSDSIINWAEGQPPSSVNDSARAMMQRVREYLADSGGSIDSSF
MVNVEDKTTFITLKTASLIEKYKNDIIIRFKSRGVNIGRTTITVNSIGEKPIYKATNAGIIPL
EGGELQTDGIYEIVYNSNVSIEDYDGWFLLNPTPLPPPKVEPFPCGFIATFAMQEMPNGW
LLCDGAVYKRKDYPQLFKAIGDKWGKDSNTTFKVPDFRGMFLRGFDNGRGLDSNRQF
AKEQQDCIKSHEHVCTIERAGEHTHNFQYDGVGWSANDIGRRNPSYHYQTITGTTQSAG
AHTHKVTISPTGERETRPVNTTVVYAIKS
SEQ ID NO: 16-amino acid sequence of a Bartonella elizabethae 35 kDa recombinant protein
MSNIYDWSLKADENAHSDNIINWSEGQPPSSVNDSARAMMQRVREYLADNGGSIDAGF
MINAEDKTTLITLKTVSPIKKYNNDIIIRFKAHGVNVGKTTIKVNSMGEKPIYKATHTGVI
SLEGGELQTDGIYEMVYNGNISTEGRDGWYLLNPTSPKVESFPTGFIATFAMQEIPTGWL
LCDGKAYKRKDYPQLFKAIGDKWGKDSDTTFKVPDFRGMFLRGFDDGRGLDDNRKFA
DEQQDSIKSHTHIGNIEESGEHTHNFQYKGVGWPTGNIGRLPNYYTYNATLQGTTDSAG
AHTHKVTLSHTGETETRPVNTTVVYAIKS
SEQ ID NO: 17-amino acid sequence of a Bartonella henselae 31 kDa recombinant protein
MNIKSLMTTSVIALISASAAQAADVIVPHEVAPTVISAPAFSWTGFYIGGQVGNFSSKVEI
TDPNKKDKLFSKDDTPKPSGFMGGIYAGSNMDLGNNMILGVETDAVWADREDAKTSS
AEAIGQDELETFRDSLKKANAAFAQGKTSDNVAAVDKHTDSLALKEKWSGATRVRIGF
TAADRIMPYVAGGVSYAQVQAVSSTKVTQAADDAEIATAQLEDKTKTLVGFTLGGGVD
FAMTDNVLLRAEYRYSDFGKKKFEKEGSEFSYKTNDFRVGVAYKF
SEQ ID NO: 18-amino acid sequence of a Bartonella quintana 31 kDa recombinant protein
MNIKSLITTSVIALVSASAAQAADVIATHEAAPVITTPTFSWTGFYIGGQIGNFTSDNKIKG
LGKETSIFTKELTPQLSGIVGGIYAGSNIDLGSGLILGVETDAIWADCEATKTSAIRTLSVP
QANSLNDEFKAAGITLKDKFSENDTMSDHYTYKAKWSGATRARIGFSAFDRVMPYFAG
GVAYARMQGMKSVSGMNAAKNKKLGGGLYDETKMMVGFTVSGGVDVAMTGNVLL
RGEYRYSDFGKKKFLNNTQEFNYKTNDFRFGVAYKF
SEQ ID NO: 19-amino acid sequence of a Bartonella vinsonii 31 kDa recombinant protein
MNIKSLITTSVIALASASAVQAADVIIPHEAAPVISAPAFSWTGFYIGGQVGSFSSKVEITE
PSKNKLNKDLTPKPSGFMGGVYAGSNIELGNNLILGVETDAVWADREETKERFSHVLEA
DELDAFKGDLTQLKASPATGKQIAAIKTGDKRTDSSTLKEKWSGATRVRIGFAAADHIM
PYVAGGVAYAQMQGTYTIKATEAKGNABIATAKLFDATKTFVGYTLGGGVDIAMTDN
VLLRAEYRYSDYGKKQFDRDKDEFSYKTNDFRVGVAYKF
SEQ ID NO: 20-amino acid sequence of a Bartonella elizabethae 31 kDa recombinant protein
MNIKFLMATSVVTLVSVSAANAADVIVPHEVAPAVVTAPSFSWTGFYLGGQIGNFSSKT
KVTIPGEDKELFKKDNTPSPSGFMGGIYAGSNVDLGSGLILGVETDAVWADREESKTAR
ESTLTKKDATFFNDALKKAKVELTGQQKFIEDDQVTETHSYKEKWSGATRVRIGFAAV
DRIMPYVAGGIAYAQVQGIQSVAGKGERAKVEQKADGGEELPEGNKQTIDLIGGTWAD
DTKTMVGFTIGGGVDFAMTDNVLLRAEYRYSDFGKKKFANDTREFNYKINDERVGVA
YKF
SEQ ID NO: 21-amino acid sequence of a Bartonella bacilliformis 31 kDa recombinant protein
MNIKCLVTASVCALISASAAQAADVMIPQEISPIISAPTFSWTGLYLGGQIGGLSGKHDFK
AIGKDSEWPFANKDLKVSGFVGGLYAGSNIDLGSGLVLGVDTDIVWVDKEGKLSSNHK
AETQDDADAFKQIFDENKIEVAKGQIKELTQNFSLKEKWAGATRVRIGFGADRIMPYVS
GGVAYTQVQAIGSAILKGTKDTGTEGGGGSASKAVRSEALDVLASGTITDEKKTLLGYT
LGAGVDFAMTDNVILRTEYRYSDFGKKKFVKDAIETNYKTNDFRVGVAYKF
SEQ ID NO: 22-amino acid sequence of a Bartonella clarridgeiae 31 kDa recombinant protein
MNMKCLMTASVVALMTAYAAQAADITVPQETGETIVVAPAFSWTGFYVGGQIGGFSSK
TSFSSRDGDDSKKWLPLHKDLWSKPSGFTGGFYTGSNVDLGNGLILGVDTDVIWSGRK
DEKTHEVTVELEDKDKNKHQEKSVSRSPVVFADGEEKEVEITKYGHTLKEKWAGATRI
RIGFAADRIMPYVAGGVAYTQLQDSFSTSVSRQKVSGAKAERADKEPSAEPNTVNTVDE
SKTMIGYTLGAGVDFAMTDNVVLRAEYRYSDFGKKKLHNDKLEVNYKANDFRVGVA
YKF
SEQ ID NO: 23-amino acid sequence of a Bartonella grahamii 31 kDa recombinant protein
MNIKSLVTTSVIAMAAASAAQAADVIIPRETAPAVISAAPSFSWTGFYIGGQVGNFSGKV
DEFDSETKKKITDKDWTPKPSGFMGGIYVGSNVDLGNGLILGVETDAVWADREDSKTH
SAVIGETGLAAFKNKLTAAEATFASGKNIDNVTKDGKRVEGTSIKEKWSGATRVRIGFA
AVDRILPYFAGGIAYTQLQVVNSLKAEGADGAEIASAKMFDQSKTMVGFTVGGGVDFA
MTDNVLLRAEYRYSDFGKKAFVKDEDKIAYKTNDFRVGVAYKF
SEQ ID NO: 24-amino acid sequence of a Bartonella koehlerae 31 kDa recombinant protein
MNIKSLMTTSVIALLSASAVQAADVIVPHEIAPAVITAPTFSWTGFYIGGQVGNFSSKVE
VTDPNTKNKLFSKDDAPKPSGFMGGVYAGSNIDLGNNVILGVETDAVWADREDSKTLS
SKAIEETKLGEFKASLTKANAVVATGKKIDDIKKEDKHTDSLVLKEKWSGATRVRIGFÅ
AADRIMPYVSGGVSYVQVQAVSSTKVTQAADGAEIVSAKFFDKTKTLVGFTLGGGVDF
AMTDNVLLRAEYRYSDFGKKKFEKDGSELSYKTNDFRVGVAYKF
SEQ ID NO: 25-amino acid sequence of a Bartonella rochalimae 31 kDa recombinant protein
MNIKHLLVACSWTFIPVSMVQAADIMKPHQSQSYQPVPVISSSNNFSWTGFYVGAQIGG
FSGEATLSPIQDSVEEDREGQGFSEIKSPKLSGFIGGIYAGSNFSLSKDLILGIDTDMVWSG
KKNTKVFDVQDMSIDMSIKEKWAGAMRMRVGFAMDRVMPYVSGGVSYVQLQNVFTA
KRLAKAVVRERRNTLPSDSEPKFLDETKILVGYSVGGGVDVAMTDNVIMRAEYRYSDF
GKKKFAKDNVDLSYKTNDFRVGVAYKF
SEQ ID NO: 26-amino acid sequence of a Bartonella washoensis 31 kDa recombinant protein
TKDETPQPSGFMGGIYAGSNVDLGNGLILGVETDAVWADRGETKTISVFSGNSSETSNA
GASQTPNAGAEEKKSISLKTTALVDAKIKNNFTFKEKWSGATRVRVGFAAADRILPYVA
GGIAYAQMQGIATVSGKDASGKTVSTKESDKTMTMVGFTIGGGVDFAMTDNVLLRAE
YRYSDFGKKKFEKDDAEFHYKINDFRVGVAYKF
SEQ ID NO: 27-amino acid sequence of a Bartonella henselae 26 kDa recombinant protein
MKKVIFKPLKSYPVKMAMIALTLLAASPITHAEESKMKNATITVTATGENQATPDMAIIN
LAVVTQDKTAQKALADNNKSMNDIINAFKNNGIQANDLQTSGLSIYQSNPNKDHEKKN
NGIVYHVSNSLTVRIRDLSNAGKIFDQAMALGVNSVHGITFTNANTKPFYQEARKKAIA
EAIEKAKTIAEAADVKLGKIIKINENDDNYYSRPHLMSKAVNANYTDTTFSSGELNYSVS
VTIVFAID
SEQ ID NO: 28-amino acid sequence of a Bartonella quintana 26 kDa recombinant protein
MTKIISQPLKSYSIKMAMITLTLLAASLPTHAEESKIKNATITVTATGESQATPDMAIINLA
IVTHDKTAQKALAANNKSMNDIVNAFKNNGIQANDLQTSGLSIYQSSPEKHREKKNTEI
LYQVSNSLTVRIRDLSNAGKIFDQAMALGVNSVNGITFTNANTKPFYQEARKKAVTAAI
EKAETIAQAANLKLGKIITINENDDNSYSRPHLMSRAAHASYADTTFSSGELNYNVSVTV
VFAID
SEQ ID NO: 29-amino acid sequence of a Bartonella vinsonii 26 kDa recombinant protein
MKKTTFQPLNHYSVRIAMVALALITSSLPIHAEESKMKNATITVTATGESQAAPDMAIIN
LAVVTEDKTAQKALADNNKSMNDIVNTFKDNGIQANDLQTSGLSIYQSSPDKPHDKKN
HEKLYHVSNSLTVRIRDLSNAGKIFDQAMALGVNSVNGITFTNADTKPLYQEARKKAIT
EAIEKAETIAQAANLKLGKIIEINENNDNYYPKPRLMSSAAHASYADTNFSGGELGYNVS
VTVVFAID
SEQ ID NO: 30-amino acid sequence of a Bartonella elizabethae 26 kDa recombinant protein
IQPLKSSLVKIAILTFTLLTTSLAVVHAYAEESKRDATITVTAIGESQATPDMAIINLAVVT
HDKTAQKALASNNQSINEIIKAFKNNGIQANDLQTSGLSIYQSSHEKKNNEKLYQVSNSL
TVRIRDLSNAGKIFDQAMALGINSVNGITFTNADTKPFYQEARKKAITEAIEKAQTIAQA
ADLKLGKIIEINEKDDYYRPTPRLMSRAADASYADTNFAGGELNYNVSVTVVFAID
SEQ ID NO: 31-amino acid sequence of a Bartonella bacilliformis 26 kDa recombinant protein
MTKAISQLFNIYWTKITILTILALLTSSLSVYAEKSTIKNSTITVTATGESQATPDMAIMNL
AVITHNKTAQKALEANNKSINNIIDSFKKEGIQEKDIQTSNLFIHYINSDKHQEQRGDENL
YQVSHSLTVRIRDLLNAGMILDQAMALGINSVRDITFINTDTKPFYTAARKQAIAEAIEK
AETLAHAASVKLGKIIEINESNDPPHLMPRLISRAQTASYADTHESSGELNYSVTVTVTFA
ID
SEQ ID NO: 32-amino acid sequence of a Bartonella clarridgeiae 26 kDa recombinant protein
MTKIIFQLLNTYRIKLSILTVLSLFATSLFINAEETIKDAPTITVTATGENQATPDMAIINLA
VVTHDTTAQKALAANNKFMNDIVNTFKNNGIQANDLQTSGLSIYQTNSDKKREKKNNE
IIYHVSNSLTVNIRDLANAGKIFDQAMALGVNSVNGITFTNADTKPLYKEARKKAIAEAI
EKAETLAQAANVKLGKIITISENNNGHYPTPRLMSRAQHARSYTDTNFSGGELNYNVSV
TITFAID
SEQ ID NO: 33-amino acid sequence of a Bartonella grahamii 26 kDa recombinant protein
VILAFTLLATSLTVRAYAEENKMKNATITVTAIGESQAAPDMAIINLAVVTYDKTAQKA
LASNNQSINDIIKAFKNDGIQANDLQTSGLSIYQSTSDKHHEKKNNEKLYHVSNSLTVRIR
DLNNAGKIFDQAMTLGVNSVNGITFTNADTKPFYQEARKKAITEAIEKAQTIAQAADLK
LGKIIEINERDDYYHPTPRLMSRAADASYENTNFAGGELTYNVSVTVVFAID
SEQ ID NO: 34-amino acid sequence of a Bartonella koehlerde 26 kDa recombinant protein
QKALADNNKSMNDIVNAFKNNGIQENDLQTSGLSIYQSNQGKHHEKKNNGNGIVYHVS
NSLTVRIRDLSNAGKIFDQAMALGVNSVHGITFTNADTKPFYQEARKQAITEAIEKAKTI
AEAANLKLGKIIKINETDDNYYSRPHLMSKTVNASYTDTAFSSGELNYNVSVTVVFAIN
SEQ ID NO: 35-amino acid sequence of a Bartonella rochalimae 26 kDa recombinant protein
MTKMIFQLLKVYRIKLSILTVLSLLTTSCFVHAEETIKNAPTITVTATGENQATPDMAIINL
AVVTHDTTAQKALAANNKFMNDILDTFKNNGIQANDLQTSGLSIYQTNSDKKQEKKNN
EITYHVSNSLTVNIRNLANAGKIFDQAMALGVNSVNGITFTNADTKPLYKEARKKAVIE
AIAKAETLAQAANVKLGKIITINESNNEYYPTPRLMSRAQSASYADTNFSGGELNYHVSV
TITFAID
SEQ ID NO: 36-amino acid sequence of a Bartonella washoensis 26 kDa recombinant protein
QEALAANNKSMNDIVNAFKSNGIQANDLQTSNLSIYQSSPDNHHEKKNKEILYRVSNSL
TVRIRDLANAGKIFDQAMALGINSVNGITFTNADMKPFYQDARKKAIAEAIEKAETIAQA
AHLKLGKIIEINENSDGYHPSPRLMSRAANASYADTNFSGGELNYSVSVTVVFTID
SEQ ID NO: 37-amino acid sequence of a Bartonella henselae 17 kDa recombinant protein
MKKYSLVTLLSLFCISHAKAQTAPLTDEYYKKALENTQKLDVAKSQTAQSIYESATQTA
NKIKDINNQLANLKADTKTKPEQLQALQIELSLLQAQLQADTLKIQSLAMIQAKDTKTK
EELREEQTQKKHEDLQKQLKEKLEKSDVRL
SEQ ID NO: 38-amino acid sequence of a Bartonella quintana 17 kDa recombinant protein
MAAYISPKERKSMKKYGLVTLLSLSCVSHTIAETALLADEYYKRALENTQKLDAAKSET
AESIYASANEITNKIKEINEKLRKAQAAEKTKPEEFQALQIELSLLQAQLQADTLKIQFLS
MIQAKNTKTKEDIREEQTQKKHKDLQEKLKEKLGNSDVRL
SEQ ID NO: 39-amino acid sequence of a Bartonella vinsonii 17 kDa recombinant protein
MKKYSLVTLLSSFFISHAVANNSKDVDEYYKQALESTQKLNVVKSETAETIFDHATQTA
KKIQEISEQLKQKQSQEKPDAEKLQTLQVELSVLQAKLQAEALKLQSLDMIQAKDSRTK
EEIREEEIKKQHEKIVQKLKEKLEKSNVPSTLGKLND
SEQ ID NO: 40-amino acid sequence of a Bartonella elizabethae 17 kDa recombinant protein
MKKYGLMTLLSFSFISHAFSQMSPDADEYYKQALENTQKLDTAKSETAETIYKTATETA
KKIKEINGQLEQLKSQTETTATETTSDADKNSNLEELQKRQALRQELQVKVSLLQADLQ
ANSLKLQALNMIQARDTKTKEELREEKEQQKHKTLQAQLKEKEEKLKEKLESTGADVR
L
SEQ ID NO: 41-amino acid sequence of a Bartonella kochlerae 17 kDa recombinant protein
MKKYSLVTLLSLFCISHAKAQIAPLPDEYYKKALENTQKLDVAKSQTAESIYESATQTAN
KIKDINDQLKTLKADTKTKPEQLQALQIELTLLQAQLQADTLKIQSLSMIQAKDTKTKEE
LREEQTQQNHKKIEEKLKEKLEKSDVRL

### DETAILED DESCRIPTION

The present disclosure provides diagnostic panels and methods for diagnosing, and treating, Bartonellosis resulting from infection by diverse Bartonella species. To assess the impact of testing limitations and to identify exposure to Bartonella species, a modified Western Blot procedure was developed and employed. The procedure, sometimes referred to herein as an “ImmunoBlot,” uses recombinant antigens from multiple Bartonella species for the serological diagnosis of Bartonella infection. As discussed in greater detail elsewhere herein, testing was conducted on humans with suspected Bartonellosis. Positive ImmunoBlots were further characterized at the species level for Bartonella henselae, Bartonella quintana, Bartonella vinsonii, and Bartonella elizabethae.

The present disclosure provides diagnostic panels and methods for quickly, easily, and accurately detecting Bartonella antibodies in a biological sample from a subject suspected of having Bartonellosis, thereby satisfying the need for such a test. Because multiple Bartonella species have pathogenic potential for Bartonellosis, it may be beneficial for tests for Bartonella species to be inclusive. That is, a test may be able to detect antibodies to multiple species from the Bartonella genus concurrently. The present disclosure provides antigenic amino acid sequences specific for various Bartonella species. The amino acid sequences of the present disclosure have high specificity and/or sensitivity for the indicated species. The inclusion of antigenic peptides that exhibit cross-reactivity across Bartonella species boundaries is also important with respect to the development of inclusive serological, or other immunologically based assays, where the goal is to detect infection, and not necessarily to identify a particular species responsible for infection. For example, the present disclosure includes immunoassays where, in the context of a single test screen, multiple Bartonella species are detectable.

The present disclosure also provides diagnostic panels and methods for diagnosing infection by one or more species of the Bartonella genus. The present disclosure is based, in part, on the discovery of species-specific amino acid sequences encoding antigenic peptides, which may also be referred to in the art as peptide antigens or antigens, as described herein. Embodiments of the present disclosure provide antigen-specific amino acid sequences for Bartonella species, including Bartonella henselae, Bartonella quintana, Bartonella vinsonii, and Bartonella elizabethae. These amino acid sequences may be used in assays to identify infection by one or more species of the Bartonella genus in samples from subjects suspected of having Bartonellosis, including but not limited to Bartonella species comprising Bartonella henselae, Bartonella quintana, Bartonella vinsonii, Bartonella elizabethae, Bartonella bacilliformis, Bartonella clarridgeiae, Bartonella grahamii, Bartonella hoehlerae, Bartonella rochalimae, and Bartonella washoensis. With the amino acid sequences of the present disclosure, identification of Bartonella infection in subject samples is performed with speed, sensitivity, and specificity at least equivalent to or greater than other current methods.

The amino acid sequences of the present disclosure may be used in diagnostic and scientific assays. Non-limiting examples of suitable assays include ImmunoBlots, line ImmunoBlots, enzyme-linked immunosorbent assay (ELISA), etc. The amino acid sequences of the present disclosure may be used for the detection of Bartonella-specific T-cells, for example with the IgXSPOT test (IGeneX, Milpitas, CA).

### Bartonella

The genus Bartonella is comprised of fastidious Gram-negative, slow growing, and facultative intracellular bacteria belonging to the Alpha-2 subgroup of the class Proteobacteria, and the Order Rhizobiales. These microorganisms are most often transmitted to humans through animal bites or scratches (i.e., cats, dogs, and other animals), by scratch inoculation of infected flea or body louse feces into the skin, and, potentially, by bites of other vectors including ants, biting flies, keds, mites, spiders, and ticks. Prior to the HIV epidemic in the early 1990s, Bartonella species were not known to have infected animals or humans in North America or throughout much of the world. Currently, at least 40 Bartonella species or subspecies have been characterized, of which 17 Bartonella species have been implicated in association with zoonotic infections in humans. Table h provides known Bartonella species, their hosts, and their vectors.

TABLE 1
Known Bartonella species, their hosts, and their vectors.

| Bartonella Species | Host (s) | Vector(s) |
|---|---|---|
| B. henselae | Cat, human, dogs, horses | Fleas, lice, |
| | | ticks, spiders |
| B. quintana | Humans, macaques, | Human body lice, |
| | cats, dogs | fleas, bed bugs |
| B. bacilliformis | Humans | Sandflies, fleas |
| B. koehlerae | Cats, dogs, humans | Fleas |
| B. vinsonii ssp. | Dogs, horses, foxes, humans | Fleas, ticks |
| berkhoffi | | |
| B. bovis | Cattle, cats, dogs, human | Biting flies, ticks |
| B. clarridgeiae | Cats, dogs | Fleas, ticks |
| B. rattimassiliensis | Rats | Fleas |
| B. tamiae | Rats, humans | Mites |
| B. tribocorum | Rats | Fleas |
| B. rousetii | Bats | Bat flies |
| B. schoenbuchensis | Cattle | Biting flies, ticks |
| B. chomelii | Cattle | Biting flies, ticks |
| B. doshiae | Rats, humans | Fleas |
| B. grahamii | Mice, humans | Fleas |
| B. birtlesii | Mice | Fleas |
| B. mayotimonensis | Bats, humans | Bat flies, fleas, ticks |
| B. elizabethae | Rats, humans, dogs | Fleas |
| B. washoensis | Dogs, humans | Fleas, ticks |
| B. rochalimae | Dogs, humans | Fleas, ticks |
| B. vinsonii ssp. | Dogs, humans | Fleas, ticks |
| arupensis | | |
| B. melophagi | Sheep, humans | Sheep keds |

### Production of Amino Acid Sequences

Amino acid sequences of the present disclosure may be naturally occurring and isolated from a natural source. Further, amino acid sequences of the present disclosure may be non-natural, synthetic sequences, such as sequences produced by recombinant technology or sequences synthesized by protein synthesizing apparatuses. As such, amino acid sequences of the present disclosure may be isolated or may be produced by recombinant technology, as is described and enabled in the literature and in commonly referred to manuals such as, for example, Short Protocols in Molecular Biology, Second Edition, F. M. Ausubel, Ed., all John Wiley & Sons, N.Y., edition as of 2008, and Sambrook, et al., Molecular Cloning: A Laboratory Manual, 3rd ed., Cold Spring Harbor Laboratory Press, Cold Spring Harbor, 2001, and as is well known to one of ordinary skill in the art.

In some embodiments, amino acid sequences of the present disclosure may be made recombinantly in a vector. As used herein, the term “vector” refers to a nucleic acid molecule capable of transporting, between different genetic environments, another nucleic acid to which it has been operatively linked. In addition to including a nucleic acid sequence encoding an amino acid sequence of the present disclosure (e.g., SEQ ID NOs: 1-41), or a variant thereof that retains the immunological binding profile of the corresponding non-variant, vectors of the present disclosure may also include a heterologous nucleic acid sequence. As used herein, a “heterologous nucleic acid sequence” is one that does not naturally occur in the organism from which the amino acid sequence of the present disclosure are derived.

The term “vector” may also refer to a virus or organism that is capable of transporting the nucleic acid molecule. One type of vector is a plasmid, a small, circular, double-stranded, extrachromosomal deoxyribonucleic acid (DNA) molecule that is physically separate from and can self-replicate independently from chromosomal DNA. Some useful vectors are those capable of autonomous replication and/or expression of nucleic acids to which they are linked.

Vectors capable of directing the expression of nucleic acids to which they are operatively linked are referred to herein as “expression vectors.” Other useful vectors, include, but are not limited to, bacterial plasmids, bacterial artificial chromosomes (BACs), cosmids, and viruses such as lentiviruses, retroviruses, adenoviruses, and phages.

Vectors useful in methods of the present disclosure may include additional sequences including, but not limited to, one or more signal sequences, one or more promoter sequences, or a combination thereof. Promoters that may be used in methods and vectors of the present disclosure include, but are not limited to, cell-specific promoters and general promoters. Non-limiting examples of promoters that can be used in vectors of the present disclosure are ubiquitous promoters such as, but not limited to, CMV, CAG, CBA, and EF1a promoters. Methods to select and use suitable promoters are within the knowledge of one skilled in the art.

Vectors useful in methods of the present disclosure may be used to express a fusion protein comprising sequences of the present disclosure (e.g., SEQ ID NOs: 1-41), and/or variants thereof that retain the immunological binding profile of the corresponding non-variants, in a cell. Expression vectors and methods of their preparation and use are within the knowledge of one skilled in the art. In some embodiments of the present disclosure, a nucleic acid sequence of an expression vector encodes a fusion protein comprising an amino acid sequence of the present disclosure, or a variant thereof that retains the immunological binding profile of the corresponding non-variant. How to prepare and utilize fusion proteins that comprise a polypeptide sequence are within the knowledge of one skilled in the art.

In some embodiments, a fusion protein comprising an amino acid sequence of the present disclosure, or a variant thereof that retains the immunological binding profile of the corresponding non-variant, may also include a tag, as described elsewhere. In some embodiments, a fusion protein comprising an amino acid sequence of the present disclosure, or a variant thereof that retains the immunological binding profile of the corresponding non-variant, may include a label, as described elsewhere herein.

In some embodiments, amino acid sequences of the present disclosure (e.g., SEQ ID NOs: 1-41), or variants thereof that retain the immunological binding profile of the corresponding non-variants, may be made recombinantly in E. coli.

### Amino Acid Sequence Homology and Variants

As used herein, a non-variant amino acid sequence is an amino acid sequence with 100% sequence homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41.

As used herein, an amino acid sequence variant, that retains the immunological binding profile of the corresponding non-variant amino acid sequence, is an amino acid sequence having at least 90%, 95%, 98%, 99%, 99.5%, or 99.9% sequence homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41.

Variants of amino acid sequences SEQ ID NOs: 1-41, which retain the immunological binding profile of the corresponding non-variant, may have conservative amino acid substitutions in conserved or non-conserved regions. A variant may have any modification(s) of a specified amino acid sequence (e.g., SEQ ID NOs: 1-41) which retain(s) the immunological binding profile of the corresponding non-variant. Such modifications may include insertions and deletions (e.g., internal or from the N- or C-terminus, or both). One skilled in the art, using no more than routine experimentation, could design and produce antigenic amino acid sequences carrying conservative amino acid substitutions in non-conserved regions, or even at non-conserved amino acid positions as identified by alignment comparisons.

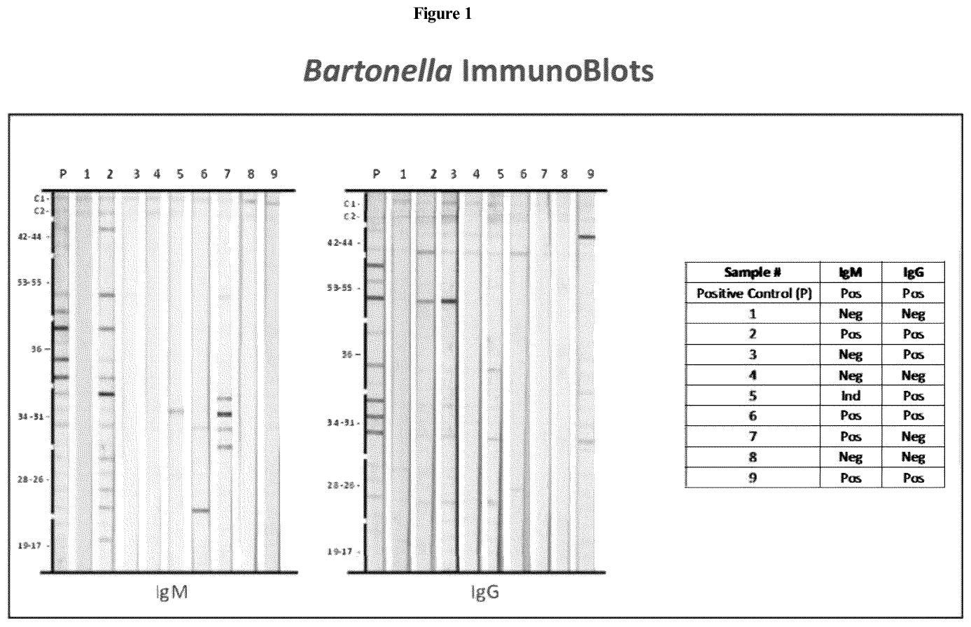

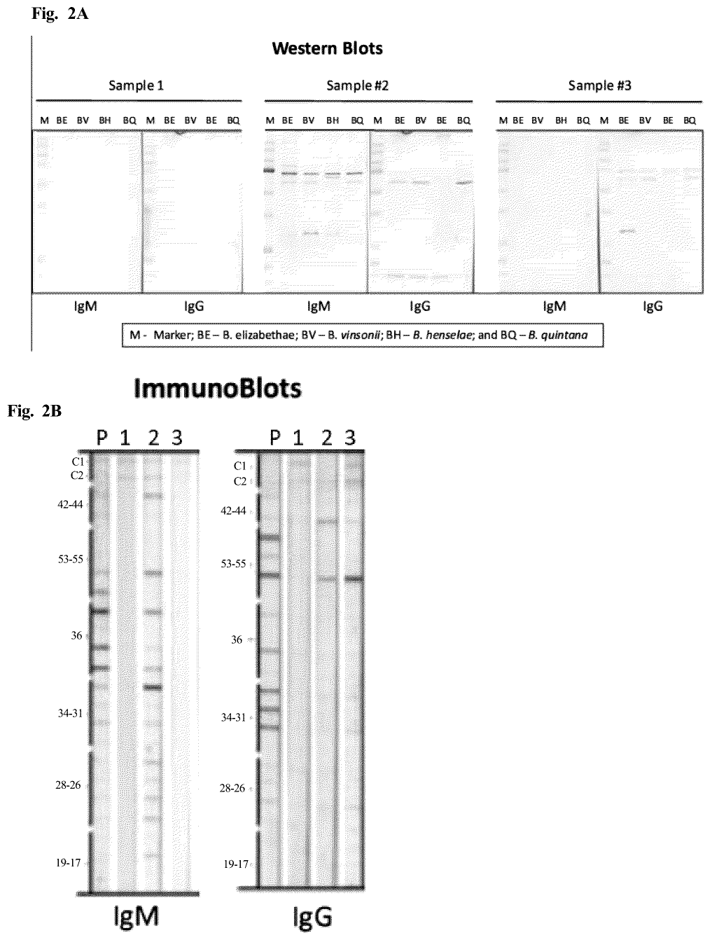

As used herein, the term “immunological binding profile” refers to the ability of a labelled, tagged, or bound amino acid sequence to be bound by antibodies present in a biological sample. Non-limiting examples of immunological binding profiles include FIGS. 1-2.

Sequences with less than 100% homology may be modified with one or more substitutions, deletions, insertions, or other modifications with respect to the amino acid sequences provided herein. Illustrative modifications include, but are not limited to, conservative amino acid substitutions, which will produce molecules having functional characteristics similar to those of the molecule from which such modifications are made. Conservative amino acid substitutions are substitutions that do not result in a significant change in the activity or tertiary structure of a selected polypeptide or protein. Such substitutions typically involve replacing a selected amino acid residue with a different residue having similar physico-chemical properties. For example, substitution of Glu for Asp is considered a conservative substitution because both are similarly sized negatively charged amino acids. Groupings of amino acids by physico-chemical properties are known to those of skill in the art. The following groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M). One of ordinary skill in the art can determine if sequences with less than 100% homology can bind naturally- or non-naturally occurring Bartonella-related antibodies, as well as the sensitivity and specificity of the antibody to the modified sequences. One of ordinary skill in the art will be able to identify sequences with significant homology to SEQ ID NOs: 1-41 of the present disclosure that give acceptable or equivalent responses in the methods of the present disclosure without undue experimentation, in view of the teachings of the present disclosure.

Nucleic acid sequences, including polynucleotides and oligonucleotides, encoding the amino acid sequences of the present disclosure, and portions thereof, may be expressed in cultured cells to provide isolatable quantities of peptides displaying biological (e.g., immunological) properties of the antigenic amino acid sequences of the present disclosure. Because of redundancy of the genetic code, multiple nucleic acid sequences may be suitable for the production of the amino acid sequences of the present disclosure. One of ordinary skill in the art will be able to determine one or more nucleic acid sequences for production of the amino acid sequences of the present disclosure. A nucleic acid sequence encoding an amino acid sequence of the present disclosure may be labelled by any suitable label known to one of ordinary skill in the art.

In this regard, nucleic acid sequences suitable for the production of the amino acid sequences of the present disclosure may be substantially homologous to naturally occurring sequences. Substantial homology of a nucleic acid sequence as used herein means that: (a) there is greater than 65%, 75%, 85%, 95%, 98%, or 99% homology with the naturally occurring sequence, or (b) the homologous nucleic acid sequence will hybridize to the compared sequence or its complementary strand under stringent conditions of temperature and salt concentration. These stringent conditions will generally be a temperature greater than about 22° C., usually greater than about 30° C. and more usually greater than about 45° C., and a salt concentration generally less than about 1 M, usually less than about 500 mM, and more usually less than about 200 mM. The combination of temperature and salt concentration is more important in defining stringency than either the temperature or the salt concentration alone. Other conditions which affect stringency include GC content of the compared sequence, extent of complementarity of the sequences, and length of the sequences involved in the hybridization, as well as the composition of buffer solution(s) used in the hybridization mixture. These and other factors affecting stringency are well described in the scientific and patent literature. One of ordinary skill in the art will be able to determine suitable conditions for determining the homology of the nucleic acid sequences encoding the antigenic amino acid sequences of the present disclosure.

Homologous nucleic acid sequences may be determined based on the nature of a nucleotide substitution in the nucleic acid sequence. For example, synonymous nucleotide substitutions, that is, nucleotide changes within a nucleic acid sequence that do not alter the encoded amino acid sequence, will be better tolerated and, therefore, may be more numerous in a particular nucleic acid sequence than non-synonymous nucleotide substitutions. One of ordinary skill in the art will be able to determine the suitable number and location of substitutions that may be allowed in a nucleic acid sequence that encodes an amino acid sequence of the present disclosure without adversely affecting the antigenicity of the encoded antigenic amino acid sequence, without undue experimentation.

### Bound Amino Acid Sequences

In some embodiments, amino acid sequences of the present disclosure (e.g., SEQ ID NOs: 1-41), and/or variants thereof that retain the immunological binding profile of the corresponding non-variants, may be bound to a substrate(s).

As used herein, a “bound” amino acid sequence is an amino acid sequence that has been immobilized on a substrate(s) in order to permit the use of the amino acid sequence in a biological test such as, for example, an immunoassay. A bound amino acid sequence may be covalently or non-covalently bound attached to a substrate(s). A bound amino acid sequence may be directly or indirectly attached to a substrate(s). Further still, the “bound” amino acid sequences of the present disclosure may be attached, directly or indirectly, to a natural surface or substance, either of which is not naturally associated with the amino acid sequence.

As used herein, a “substrate” refers to a non-natural surface or substance, or a natural surface or substance that is not naturally associated with an amino acid sequence of the present disclosure (e.g., SEQ ID NOs: 1-41), or variant thereof that retains the immunological binding profile of the corresponding non-variant. Non-limiting examples of substrates include, but are not limited to nitrocellulose, nylon, polyvinylidene difluoride (PVDF), magnetic beads, agarose (e.g., beads), plastic, and metal.

The number of substrates used, in a particular diagnostic panel of the present disclosure, may depend on the number of amino acid sequences of the diagnostic panel. For example, when the diagnostic panel is a line ImmunoBlot, more than one nitrocellulose sheet may be used as part of the diagnostic panel. For example, a first portion of the amino acid sequences may be applied to a first nitrocellulose sheet, a second portion of the amino acid sequences may be applied to a second nitrocellulose sheet, etc. Notwithstanding, the more than one nitrocellulose sheet may be analyzed together to determine a result of the line ImmunoBlot.

Linking agents known to those of ordinary skill in the art may be used to aid and/or enhance binding of the amino acid sequences of the present disclosure (e.g., SEQ ID NOs: 1-41), and/or variants thereof that retain the immunological binding profile of the corresponding non-variants, to a substrate(s).

### Tagged Amino Acid Sequences

In some embodiments, amino acid sequences of the present disclosure (e.g., SEQ ID NOs: 1-41), and variants thereof that retain the immunological binding profile of the corresponding non-variants, may be tagged. As used herein, a “tagged” amino acid sequence is an amino acid sequence comprising a tag. As used herein, a “tag” refers to a peptide sequence that is a physical part of or chemically bound (e.g., covalently, or via hydrogen or ionic bonding) to an amino acid sequence of the present disclosure, where the peptide sequence can be detected through microscopy of other means of detection to enable visualization and/or localization of the corresponding amino acid sequence.

Numerous tags are well known in the art and routine methods can be used to include such tags in amino acid sequences and methods of the present disclosure. A non-limiting list tags includes, but is not limited to, affinity tags, chromatography tags, epitope tags, enzyme conjugate tags, and fluorescence tags.

An affinity tag enables purification of its conjugated-to moiety from a biological sample using an affinity technique. Example affinity tags include, but are not limited to, chitin binding protein (CBP), maltose binding protein (MBP), strep-tag, poly-histidine tag (His), and glutathione-S transferase (GST).

A chromatography tag alters one or more chromatographic properties of the tagged amino acid sequence, thereby affording resolution of the tagged amino acid sequence across a particular separation technique. Example chromatography tags include, but are not limited to, polyanionic amino acids, such as FLAG-tag and polyglutamate tag.

An epitope tag is a peptide sequence having high affinity for one or more antibodies usable in antibody purification. Example epitope tags include, but are not limited to, ALFA tag, Vs tag, Myc tag, human influenza hemagglutinin (HA) tag, Spot tag, T₇ tag, FLAG tag, and NE tag.

An enzyme conjugate tag is an enzyme usable to conjugate antibodies, streptavidin, and other proteins using a wide variety of mechanisms. Typically, a substrate needs to be added to the enzyme conjugate to generate the appropriate colorimetric, chemiluminescent, or fluorescent signal output. Example enzymes conjugate tags include but are not limited to horseradish peroxidise (HRP), alkaline phosphatise (AP), glucose oxidase, and β-galactosidase.

A fluorescence tag enables a visual readout on the tagged amino acid. An example fluorescence tag is green fluorescent protein (GFP).

Because tagged amino acid sequences of the present disclosure must retain immunological specificity for antibodies, particular tags must be carefully selected to avoid, for example, steric interference with such immunological specificity. One skilled in the art can select tags using no more than routine experimentation.

### Labelled Amino Acid Sequences

In some embodiments, amino acid sequences of the present disclosure (e.g., SEQ ID NOs: 1-41), and variants thereof that retain the immunological binding profile of the corresponding non-variants, may be labelled. As used herein, a “labelled” amino acid sequence is an amino acid sequence comprising a label. As used herein, a “label” refers to a detectable moiety that is a physical part of or chemically bound (e.g., covalently, or via hydrogen or ionic bonding) to an amino acid sequence of the present disclosure, where the detectable moiety can be detected through microscopy of other means of detection to enable visualization and/or localization of the corresponding amino acid sequence.

Numerous labels are well known in the art and routine methods can be used to include such labels in amino acid sequences and methods of the present disclosure. A non-limiting list labels includes, but is not limited to, stable isotopes, mass tags, and fluorophores.

Stable isotopes enable measuring and differentiation of corresponding labelled amino acid sequences using mass spectrometry. Example stable isotopes include, but are not limited to ²H, ¹H, ¹³C, ¹⁴C, ¹⁵N, ³²P, ³⁵S, and ¹²⁵I.

Mass tags enable measuring and differentiation of corresponding labelled amino acid sequences using mass spectrometry. Mass tags include tandem mass tags, which are chemical labels that facilitate sample multiplexing in mass spectrometry-based quantification and identification of biological macromolecules. Tandem mass tags belong to a family of reagents referred to as isobaric mass tags, which are a set of molecules with the same mass, but which yield reporter ions of differing mass after fragmentation. The relative ratio of the measured reporter ions represents the relative abundance of the tagged molecule. Example tandem mass tags include, but are not limited to, TMTzero, TMTduplex, TMTsixplex, TMT 10-plex, TMTpro, and TMTpro Zero.

Fluorophores are chemical compounds that re-emit light upon excitation and are detected with fluorescence imagers. Example fluorophores that may be used in accordance with the present disclosure include organic dyes such as fluorescein, rhodamine, and aminomethylcoumarin acetate. Such small molecules, when used as a label, are less likely to present steric hindrance in connection with antibody binding.

### Diagnostic Panels

In some embodiments, a diagnostic panel of the present disclosure includes individually identifiable labelled, tagged, or bound amino acid sequences, where the amino acid sequences comprise SEQ ID NOs: 1-41, and/or one or more variants thereof that retain the immunological binding profile of the corresponding non-variant. Such a diagnostic panel may be used to detect infection by Bartonella, at the genus level, as well as optionally be used to detect infection by the specific species Bartonella henselae, Bartonella quintana, Bartonella vinsonii, and Bartonella elizabethae.

In some embodiments, a diagnostic panel of the present disclosure includes individually identifiable labelled, tagged, or bound amino acid sequences, where the amino acid sequences comprise SEQ ID NOs: 1-12, and/or one or more variants thereof that retain the immunological binding profile of the corresponding non-variant. Such a diagnostic panel may be used to detect infection by Bartonella, at the genus level.

In some embodiments, a diagnostic panel of the present disclosure includes individually identifiable labelled, tagged, or bound amino acid sequences, where the amino acid sequences comprise SEQ ID NOs: 13-20 and 27-30, and/or one or more variants thereof that retain the immunological binding profile of the corresponding non-variant. Such a diagnostic panel may be used to detect infection by the specific species Bartonella henselae, Bartonella quintana, Bartonella vinsonii, and Bartonella elizabethae.

In some embodiments, a diagnostic panel of the present disclosure includes individually identifiable labelled, tagged, or bound amino acid sequences, where the amino acid sequences comprise SEQ ID NOs: 13, 17, and 27, and/or one or more variants thereof that retain the immunological binding profile of the corresponding non-variant. Such a diagnostic panel may be used to detect infection by Bartonella henselae.

In some embodiments, a diagnostic panel of the present disclosure includes individually identifiable labelled, tagged, or bound amino acid sequences, where the amino acid sequences comprise SEQ ID NOs: 14, 18, and 28, and/or one or more variants thereof that retain the immunological binding profile of the corresponding non-variant. Such a diagnostic panel may be used to detect infection by Bartonella quintana.

In some embodiments, a diagnostic panel of the present disclosure includes individually identifiable labelled, tagged, or bound amino acid sequences, where the amino acid sequences comprise SEQ ID NOs: 15, 19, and 29, and/or one or more variants thereof that retain the immunological binding profile of the corresponding non-variant. Such a diagnostic panel may be used to detect infection by Bartonella vinsonii.

In some embodiments, a diagnostic panel of the present disclosure includes individually identifiable labelled, tagged, or bound amino acid sequences, where the amino acid sequences comprise SEQ ID NOs: 16, 20, and 30, and/or one or more variants thereof that retain the immunological binding profile of the corresponding non-variant. Such a diagnostic panel may be used to detect infection by Bartonella elizahethae.

In some embodiments, a diagnostic panel of the present disclosure includes individually identifiable labelled, tagged, or bound amino acid sequences, where the amino acid sequences consist of SEQ ID NOs: 1-41, and/or one or more variants thereof that retain the immunological binding profile of the corresponding non-variant. Such a diagnostic panel may be used to detect infection by Bartonella, at the genus level, as well as optionally be used to detect infection by the specific species Bartonella henselae, Bartonella quintana, Bartonella vinsonii, and Bartonella elizabethae.

In some embodiments, a diagnostic panel of the present disclosure includes individually identifiable labelled, tagged, or bound amino acid sequences, where the amino acid sequences consist of SEQ ID NOs: 1-12, and/or one or more variants thereof that retain the immunological binding profile of the corresponding non-variant. Such a diagnostic panel may be used to detect infection by Bartonella, at the genus level.

In some embodiments, a diagnostic panel of the present disclosure includes individually identifiable labelled, tagged, or bound amino acid sequences, where the amino acid sequences consist of SEQ ID NOs: 13-20 and 27-30, and/or one or more variants thereof that retain the immunological binding profile of the corresponding non-variant. Such a diagnostic panel may be used to detect infection by the specific species Bartonella henselae, Bartonella quintana, Bartonella vinsonii, and Bartonella elizabethae.

As used herein, “consist of”, “consists of”, and “consisting of”, when referring to amino acid sequences of a diagnostic panel, refers to a diagnostic panel having amino acid sequences limited to the specified amino acid sequences. That is, the diagnostic panel may have components other than the specific amino acid sequences, but the amino acid sequences included in the diagnostic panel are limited to those specified. In other words, the amino sequences are limited to those specified, but the diagnostic panel is not precluded from having other non-amino acid sequence components.

### Assays and Methods of Detection

Amino acid sequences of the present disclosure exhibit antigenic activity with respect to various antibodies of Bartonella origin, as shown by the Examples provided herein below. In other words, the antibodies of Bartonella origin show specificity for the amino acid sequences of the present disclosure. Specificity for said amino acid sequences (i.e., antibody specificity) is the property of antibodies that enables them to react preferentially with some antigenic determinants and not with others. Specificity is dependent on chemical composition, physical forces and molecular structure at the binding site. Sensitivity is how strongly the antibody binds to the antigenic determinant. One of ordinary skill in the art can easily determine specificity and sensitivity of an antibody for a particular amino acid sequence using standard affinity assays, such as immunoblotting, Ouchterlony assays, titer assays, etc.

The Bartonella antibodies may be detected by, for example, immunoblotting, Elispot, ELISA, Western blotting, lateral flow assay, or any other appropriate immunoassay known to one of ordinary skill in the art, all of which may be referred to herein separately as a “diagnostic panel.” These techniques are known to one of ordinary skill in the art and procedures can be found in common technical references. While similar, each of these techniques has its advantages and disadvantages. Other suitable techniques may be known to those of skill in the art and are incorporated herein.

To assess the impact of testing limitations and to determine levels of exposure to Bartonella species, a modified Western blot procedure, the line ImmunoBlot, was developed and employed as described in the Examples provided herein below. According to the present disclosure, a line ImmunoBlot uses recombinant antigens from multiple Bartonella species (i.e., amino acid sequences of the present disclosure) for serological identification and diagnosis of Bartonella infection in biological samples (e.g., serum) from subjects with suspected Bartonella infection. Infection with more than one Bartonella species is possible and may occasionally be observed.

Western blotting can involve separating proteins by electrophoresis and then transferring to nitrocellulose or other solid media (e.g., polyvinylidene fluoride or PVDF-membrane and nylon membrane).

Immunoblotting can involve applying proteins to a solid media manually or by machine. Preferably, the proteins are applied in straight lines or spots and dried, binding them to the solid support medium (e.g., nitrocellulose). The bound proteins are then exposed to a biological sample or samples suspected of having antibodies specific for the target proteins. With this procedure, a known antibody can be used to determine if a protein is present in a sample, such as when the proteins of lysed cells are separated by electrophoresis and transferred to the solid medium. Western blotting allows for the identification of proteins by size as well as by specificity for a specific antibody.

In immunoblotting, known proteins can be bound to the solid medium and biological samples, such as samples from subjects suspected of having a Bartonella infection, can be tested for the presence of specific antibodies in the sample by contacting the bound protein with the biological sample. An antibody that binds the target protein is usually referred to as the primary antibody. A secondary antibody (e.g., a rabbit-anti-human IgG antibody), specific for conserved regions of the primary antibody, may be used to detect any bound primary antibodies. The secondary antibody is usually labelled with a detectable moiety for visualization. Non-limiting examples of suitable labels include, for example, chromophores such as biotin, radioactive moieties and enzymes such as alkaline phosphatase, etc. The use of these and other materials for the visualization of antibodies are well known to one of ordinary skill in the art.

The ELISPOT method can detect human T cells that respond to Bartonella-specific antigens in vitro. In an ELISPOT assay, the surfaces of PVDF membrane in a 96-well microtiter plate are coated with capture antibody that binds, for example, anti-Interferon gamma (IFNγ) or other cytokine-specific antibody. During the cell incubation and stimulation step, the T cells isolated from subject whole blood are seeded into the wells of the plate along with an amino acid sequence(s) of the present disclosure, and form substantially a monolayer on the membrane surface of the well. Upon stimulation of any antigen-specific cells with one or more of the amino acid sequences of the present disclosure, they are activated, and they release the IFN-γ, which is captured directly on the membrane surface by the immobilized antibody. The IFNγ is thus “captured” in the area directly surrounding the secreting cell, before it has a chance to diffuse into the culture media, or to be degraded by proteases and bound by receptors on bystander cells. Subsequent detection steps visualize the immobilized IFNγ as an ImmunoSpot; essentially the secretory footprint of the activated cell.

For a specific example of an ELISPOT test, each well of the plate is coated with a purified cytokine-specific antibody specific for the test or cell being detected. A subject's (i.e., a subject suspected of having a Bartonella infection) T cells are isolated and cultured in each well and stimulated with recombinant antigens of one or more sequences of the present disclosure. Bartonella-positive cells secrete cytokine in response to stimuli, which is captured by the antibody coated in the well and further detected by ELISA.

ELISA assays are also used to detect antigens. The ELISA assay can permit the quantification of a specific protein in a mix of proteins (e.g., a lysate) or determine if a peptide is present in a sample. Likewise, ELISA assays can be used to determine if a specific antibody is present by using a specific antigen as a target. As used with the present disclosure, target amino acid sequence(s) are attached to a surface. Then, if present in the biological sample being tested, the reactive antibody can bind to the antigen. A secondary antibody linked to an enzyme is added, and, in the final step, a substance containing the enzyme's substrate is added. The subsequent reaction produces a detectable signal, most commonly a color change in the substrate.

Lateral flow assays, also referred to by a variety of other names that include but are not limited to lateral flow tests, lateral flow devices, lateral flow immunoassays, lateral flow immunochromatographic assays, and rapid tests, are a further example type of diagnostic panel of the present disclosure. Lateral flow assays are simple, versatile, paper-based platforms for detecting and/or quantifying the presence of one or more analytes, such as an antigen, in a mixture, such as a liquid sample. Lateral flow assays may be qualitative or quantitative. In a lateral flow assay, a sample containing one or more analytes of interest is applied to an adsorbent sample pad and is drawn via capillary action through various zones of polymeric test strips to which are attached molecules that can interact with the analyte(s). The sample migrates to the conjugate release pad, which contains molecules that specifically bind to the analyte(s) of interest and are conjugated to fluorescent, colored, or otherwise detectable particles. Finally, the sample, including the bound analyte(s) migrates into the detection zone. Within the porous membrane of the detection zone are biological components such as antibodies or antigens, that are immobilized in lines and that will react with the detectable particles. Lateral flow assays typically have a control line for confirming sample flow through the strip and one or more test lines for detecting the presence of the analyte(s) of interest. The results may be read by eye or with a machine capable of reading and interpreting the results. A lateral flow assay may be designed as a direct or “sandwich” assay, in which the presence of a colored line at the test line position indicates a positive test, or as a competitive assay, in which the absence of a colored line indicates a positive test. Direct and competitive assays may be multiplexed.

### Methods of Detecting Infection by One or More Bartonella Species

The present disclosure provides methods of quickly and accurately detecting infection by one or more Bartonella species. In other words, the present disclosure provides methods for detecting Bartonella antibodies, in a biological sample from a subject suspected of having infection caused by one or more Bartonella species, having specificity for amino acid sequences of the present disclosure.

Methods of the present disclosure may comprise providing or obtaining a biological sample (e.g., blood or saliva) obtained from a subject suspected of having infection caused by one or more Bartonella species

Methods of the present disclosure may further comprise contacting the biological sample with a diagnostic panel of the present disclosure. For example, the diagnostic panel may comprise amino acid sequences comprising or consisting of SEQ ID Nos: 1-41, or one or more variants thereof that retain the immunological binding profile of the corresponding non-variant. For further example, the diagnostic panel may comprise amino acid sequences comprising or consisting of SEQ ID Nos: 1-12, or one or more variants thereof that retain the immunological binding profile of the corresponding non-variant.

Methods of the present disclosure may further comprise, after contacting the biological sample with the diagnostic panel, determining the biological sample is positive for Bartonella infection. Determining the biological sample is positive for Bartonella infection may comprise determining a positive immunobinding reaction that indicates the presence of one or more Bartonella antibodies in the biological sample.

In methods of the present disclosure, any primary antibody, bound to an amino acid sequence of the present disclosure, may be detected with anti-human antibodies, such as IgM-, IgG-, and IgA-class antibodies, used as the secondary antibody, and the secondary antibody may linked to, conjugated to, or otherwise associated with a detectable moiety (e.g., chromophores, radioactivity moieties and enzymes or other detectable moiety known to one of ordinary skill in the art). In some embodiments, the detectable moiety comprises alkaline phosphatase. In some embodiments, the detectable moiety comprises biotin.

In some embodiments, when the diagnostic panel comprises amino acid sequences comprising or consisting of SEQ ID Nos: 1-41, a biological sample may be determined to be positive for Bartonella infection when a positive immunobinding reaction with IgM-, IgG-, or IgA-class secondary antibodies is detected for at least one of SEQ ID Nos: 1-12 and at least one of SEQ ID Nos: 13-41.

In some embodiments, when the diagnostic panel comprises amino acid sequences comprising or consisting of SEQ ID Nos: 1-41, a biological sample may be determined to be positive for Bartonella infection when a positive immunobinding reaction with IgM-, IgG-, or IgA-class secondary antibodies (i.e., linked to, conjugated to, or otherwise associated with a detectable moiety) is detected for at least one of SEQ ID Nos: 1-4, at least one of SEQ ID Nos: 5-8, and at least one of SEQ ID Nos: 9-12.

In some embodiments, when the diagnostic panel comprises amino acid sequences comprising or consisting of SEQ ID Nos: 1-12, a biological sample may be determined to be positive for Bartonella infection when a positive immunobinding reaction with IgM-, IgG-, or IgA-class secondary antibodies (i.e., linked to, conjugated to, or otherwise associated with a detectable moiety) is detected for at least one of SEQ ID Nos: 1-4, at least one of SEQ ID Nos: 5-8, and at least one of SEQ ID Nos: 9-12.

Non-limiting examples of combinations of sequences 1-12 that may be included in a diagnostic panel of the invention are shown in Table 2. In some embodiments, a diagnostic panel of the invention comprises a combination of sequences shown in Table 2.

TABLE 2
A diagnostic panel of the invention may
comprise a combination of sequences indicated.
Combinations of Amino Acids

| SEQ ID NO | SEQ ID NO | SEQ ID NO |
|---|---|---|
| 1 | 5 | 9 |
| 1, 2 | 5 | 9 |
| 1, 2, 3 | 5 | 9 |
| 1, 2, 3, 4, | 5 | 9 |
| 1 | 5, 6 | 9 |
| 1, 2 | 5, 6 | 9 |
| 1, 2, 3 | 5, 6 | 9 |
| 1, 2, 3, 4, | 5, 6 | 9 |
| 1 | 5, 6, 7 | 9 |
| 1, 2 | 5, 6, 7 | 9 |
| 1, 2, 3 | 5, 6, 7 | 9 |
| 1, 2, 3, 4, | 5, 6, 7 | 9 |
| 1 | 5, 6, 7, 8 | 9 |
| 1, 2 | 5, 6, 7, 8 | 9 |
| 1, 2, 3 | 5, 6, 7, 8 | 9 |
| 1, 2, 3, 4, | 5, 6, 7, 8 | 9 |
| 1 | 5 | 9, 10 |
| 1, 2 | 5 | 9, 10 |
| 1, 2, 3 | 5 | 9, 10 |
| 1, 2, 3, 4, | 5 | 9, 10 |
| 1 | 5, 6 | 9, 10 |
| 1, 2 | 5, 6 | 9, 10 |
| 1, 2, 3 | 5, 6 | 9, 10 |
| 1, 2, 3, 4, | 5, 6 | 9, 10 |
| 1 | 5, 6, 7 | 9, 10 |
| 1, 2 | 5, 6, 7 | 9, 10 |
| 1, 2, 3 | 5, 6, 7 | 9, 10 |
| 1, 2, 3, 4, | 5, 6, 7 | 9, 10 |
| 1 | 5, 6, 7, 8 | 9, 10 |
| 1, 2 | 5, 6, 7, 8 | 9, 10 |
| 1, 2, 3 | 5, 6, 7, 8 | 9, 10 |
| 1, 2, 3, 4, | 5, 6, 7, 8 | 9, 10 |
| 1 | 5 | 9, 10, 11 |
| 1, 2 | 5 | 9, 10, 11 |
| 1, 2, 3 | 5 | 9, 10, 11 |
| 1, 2, 3, 4, | 5 | 9, 10, 11 |
| 1 | 5, 6 | 9, 10, 11 |
| 1, 2 | 5, 6 | 9, 10, 11 |
| 1, 2, 3 | 5, 6 | 9, 10, 11 |
| 1, 2, 3, 4, | 5, 6 | 9, 10, 11 |
| 1 | 5, 6, 7 | 9, 10, 11 |
| 1, 2 | 5, 6, 7 | 9, 10, 11 |
| 1, 2, 3 | 5, 6, 7 | 9, 10, 11 |
| 1, 2, 3, 4, | 5, 6, 7 | 9, 10, 11 |
| 1 | 5, 6, 7, 8 | 9, 10, 11 |
| 1, 2 | 5, 6, 7, 8 | 9, 10, 11 |
| 1, 2, 3 | 5, 6, 7, 8 | 9, 10, 11 |
| 1, 2, 3, 4, | 5, 6, 7, 8 | 9, 10, 11 |
| 1 | 5 | 9, 10, 11, 12 |
| 1, 2 | 5 | 9, 10, 11, 12 |
| 1, 2, 3 | 5 | 9, 10, 11, 12 |
| 1, 2, 3, 4, | 5 | 9, 10, 11, 12 |
| 1 | 5, 6 | 9, 10, 11, 12 |
| 1, 2 | 5, 6 | 9, 10, 11, 12 |
| 1, 2, 3 | 5, 6 | 9, 10, 11, 12 |
| 1, 2, 3, 4, | 5, 6 | 9, 10, 11, 12 |
| 1 | 5, 6, 7 | 9, 10, 11, 12 |
| 1, 2 | 5, 6, 7 | 9, 10, 11, 12 |
| 1, 2, 3 | 5, 6, 7 | 9, 10, 11, 12 |
| 1, 2, 3, 4, | 5, 6, 7 | 9, 10, 11, 12 |
| 1 | 5, 6, 7, 8 | 9, 10, 11, 12 |
| 1, 2 | 5, 6, 7, 8 | 9, 10, 11, 12 |
| 1, 2, 3 | 5, 6, 7, 8 | 9, 10, 11, 12 |
| 1, 2, 3, 4, | 5, 6, 7, 8 | 9, 10, 11, 12 |

In embodiments where the diagnostic panel comprises amino acid sequences comprising or consisting of SEQ ID Nos: 1-41 and it is determined that the sample is positive for Bartonella infection, the one or more particular species, causing the Bartonella infection, may be identified. For example, the biological sample may be determined to be positive for Bartonella henselae infection when a positive immunobinding reaction with IgM-, IgG-, or IgA-class secondary antibodies (i.e., linked to, conjugated to, or otherwise associated with a detectable moiety) is detected for at least one of SEQ ID Nos: 13, 17, and 27. For further example, the biological sample may be determined to be positive for Bartonella quintana infection when a positive immunobinding reaction with IgM-, IgG-, or IgA-class secondary antibodies (i.e., linked to, conjugated to, or otherwise associated with a detectable moiety) is detected for at least one of SEQ ID Nos: 14, 18, and 28. As another example, the biological sample may be determined to be positive for Bartonella vinsonii infection when a positive immunobinding reaction with IgM-, IgG-, or IgA-class secondary antibodies (i.e., linked to, conjugated to, or otherwise associated with a detectable moiety) is detected for at least one of SEQ ID Nos: 15, 19, and 29. For further example, the biological sample may be determined to be positive for Bartonella elizabethae infection when a positive immunobinding reaction with IgM-, IgG-, or IgA-class secondary antibodies (i.e., linked to, conjugated to, or otherwise associated with a detectable moiety) is detected for at least one of SEQ ID Nos: 16, 20, and 30.

### Methods of Detecting Infection by One or More Particular Bartonella Species

The present disclosure also provides methods of quickly and accurately detecting infection by one or more particular Bartonella species. Such methods of the present disclosure may comprise providing or obtaining a biological sample (e.g., blood or saliva) obtained from a subject known to having a Bartonella infection.

Methods of the present disclosure may further comprise contacting the biological sample with a diagnostic panel of the present disclosure. For example, the diagnostic panel may comprise amino acid sequences comprising or consisting of SEQ ID Nos: 13-20 and 27-30, or one or more variants thereof that retain the immunological binding profile of the corresponding non-variant. For further example, the diagnostic panel may comprise amino acid sequences comprising or consisting of SEQ ID Nos: 13, 17, and 27, or one or more variants thereof that retain the immunological binding profile of the corresponding non-variant. As another example, the diagnostic panel may comprise amino acid sequences comprising or consisting of SEQ ID Nos: 14, 18, and 28, or one or more variants thereof that retain the immunological binding profile of the corresponding non-variant. For further example, the diagnostic panel may comprise amino acid sequences comprising or consisting of SEQ ID Nos: 15, 19, and 29, or one or more variants thereof that retain the immunological binding profile of the corresponding non-variant. As another example, the diagnostic panel may comprise amino acid sequences comprising or consisting of SEQ ID Nos: 16, 20, and 30, or one or more variants thereof that retain the immunological binding profile of the corresponding non-variant. For further example, the diagnostic panel may comprise amino acid sequences comprising or consisting of SEQ ID Nos: 13, 17, and 27, or one or more variants thereof that retain the immunological binding profile of the corresponding non-variant.

Methods of the present disclosure may further comprise, after contacting the biological sample with the diagnostic panel, determining the biological sample is positive for at least one of Bartonella henselae infection, Bartonella quintana infection, Bartonella vinvonii infection, and Bartonella elizabethae infection. Determining the biological sample is positive for at least one of Bartonella henselae infection, Bartonella quintana infection, Bartonella vinvonii infection, and Bartonella elizabethae infection may comprise determining a positive immunobinding reaction that indicates the presence of at least one of Bartonella henselae antibodies, Bartonella quintana antibodies, Bartonella vinvonii antibodies, and Bartonella elizabethae antibodies in the biological sample.

For example, the biological sample may be determined to be positive for Bartonella henselae infection when a positive immunobinding reaction with IgM-, IgG-, or IgA-class secondary antibodies (i.e., linked to, conjugated to, or otherwise associated with a detectable moiety) is detected for at least one of SEQ ID Nos: 13, 17, and 27. For further example, the biological sample may be determined to be positive for Bartonella quintana infection when a positive immunobinding reaction with IgM-, IgG-, or IgA-class secondary antibodies (i.e., linked to, conjugated to, or otherwise associated with a detectable moiety) is detected for at least one of SEQ ID Nos: 14, 18, and 28. As another example, the biological sample may be determined to be positive for Bartonella vinsonii infection when a positive immunobinding reaction with IgM-, IgG-, or IgA-class secondary antibodies (i.e., linked to, conjugated to, or otherwise associated with a detectable moiety) is detected for at least one of SEQ ID Nos: 15, 19, and 29. For further example, the biological sample may be determined to be positive for Bartonella elizabethae infection when a positive immunobinding reaction with IgM-, IgG-, or IgA-class secondary antibodies (i.e., linked to, conjugated to, or otherwise associated with a detectable moiety) is detected for at least one of SEQ ID Nos: 16, 20, and 30.

### Biological Samples

As used herein, the term “biological sample” includes, but is not limited to, whole blood, serum, plasma, joint fluid, cerebrospinal fluid (CSF), urine, saliva, and mucus.

### Subjects

As used herein, a subject may be an animal, such as a mammal or a non-mammal. Non-limiting examples of mammalian subjects include primates (including but not limited to humans), rodents (including but not limited to mice, rats, squirrels, chipmunks, prairie dogs), lagomorphs, deer, canids (including but not limited to dogs, foxes, coyotes, and wolves), felids (including but not limited to domestic cats, bobcats, cougars, and other wild cats), bears, horses, cows, sheep, goats, and pigs. Non-limiting examples of non-mammalian subjects include birds, amphibians, lizards, insects, and arthropods.

### Treatment of Bartonella Infection

In some embodiments, the present disclosure involves treating Bartonella infection of a subject. As used herein, the terms “treatment,” “treat,” and “treating” refer to reversing, alleviating, or inhibiting the progress of Bartonella infection.

Treatment of Bartonella infection may involve the administration of one or more compounds, or one or more pharmaceutical compositions thereof. As used herein, the terms “administer,” “administering,” and “administration” refer to implanting, absorbing, ingesting, injecting, inhaling, or otherwise introducing one or more compounds for treating Bartonella infection, or one or more pharmaceutical compositions thereof.

A dosage level for a compound for treating Bartonella infection, or a pharmaceutical composition thereof, is within the knowledge of one skilled in the art or can be determined by one skill in the art using routine experimentation. A compound for treating Bartonella infection, or pharmaceutical composition thereof, may be administered to a subject in a pharmaceutically effective dose.

A variety of administration routes are available. The particular delivery mode selected will depend upon the particular condition being treated and the dosage required for therapeutic efficacy. Methods of this disclosure, generally speaking, may be practiced using any mode of administration that is medically acceptable, meaning any mode that produces effective levels of treatment without causing clinically unacceptable adverse effects. In some embodiments, a compound for treating Bartonella infection, or pharmaceutical composition thereof, may be administered via an oral, enteral, mucosal, percutaneous, and/or parenteral route. The term “parenteral” includes subcutaneous, intrathecal, intravenous, intramuscular, intraperitoneal, and intrasternal injection, as well as infusion techniques. Other routes include, but are not limited to, nasal (e.g., via a gastro-nasal tube), dermal, vaginal, rectal, and sublingual. Delivery routes of the present disclosure may include intrathecal, intraventricular, or intracranial. In some embodiments, a compound for treating Bartonella infection, or pharmaceutical composition thereof, may be placed within a slow-release matrix and administered by placement of the matrix in the subject.

A compound for treating Bartonella infection, or pharmaceutical composition thereof, may be administered in a formulation, which may be administered in a pharmaceutically acceptable solution, which may routinely contain pharmaceutically acceptable concentrations of salt, buffering agents, preservatives, compatible carriers, adjuvants, and optionally other therapeutic ingredients.

According to methods of the present disclosure, Bartonella infection may be treated using a pharmaceutical composition. In general, a pharmaceutical composition is a composition comprising a pharmaceutically active ingredient and one or more pharmaceutically acceptable carrier. Pharmaceutically acceptable carriers are well known to the skilled artisan and may be selected and utilized using routine methods. As used herein, a pharmaceutically acceptable carrier means a non-toxic material that does not interfere with the effectiveness of the biological activity of the pharmaceutically active ingredient. Pharmaceutically acceptable carriers may include diluents, fillers, salts, buffers, stabilizers, solubilizers, and other materials that are well-known in the art.

In some embodiments, a method of the present disclosure may comprise treating the Bartonella infection of a subject using one or more commercially available medications.

In situations where the one or more species causing the Bartonella infection are specifically identified (e.g., Bartonella henselae, Bartonella quinlana, Bartonella vinsonii, and Bartonella elizahethae), a treatment(s) specific to the one or more identified species may be administered.

### EXAMPLES

### Example 1. Detection of Bartonella Infection in Subject Samples

### Method

A total of 41 Bartonella recombinant proteins derived from Bartonella species Bartonella hensealae, Bartonella quintana, Bartonella elizabethae, Bartonella vinsonii, Bartonella vinsonii, Bartonella bacilliformis, Bartonella clarridgeiae, Bartonella grahamii, Bartonella koehlerae, Bartonella rochalimae, and Bartonella washoensis (listed in Table 3) were used to prepare antigen strips for Bartonella ImmunoBlots as described by Liu S et al., “Pilot Study of Immunoblots with Recombinant Borrelia burgdorferi Antigens for Laboratory Diagnosis of Lyme Disease,” Healthcare (Basel) 2018 Aug. 14; 6(3)-99 doi: 10.3390/healthcare6030099, which is incorporated herein by reference in its entirety. Protein sequences were from published data. Recombinant antigens were prepared by cloning the hybrid gene constructs or portions of the selected genes into pET vectors, and then expressing the proteins in Escherichia coli (GenScript, Piscataway, NJ, USA). The E. coli-produced recombinant Bartonella proteins were then purified using metal affinity chromatography followed by gel filtration. All the recombinant proteins were >90% pure by Coomassie blue staining after SDS PAGE.

TABLE 3
List of Recombinant Proteins.

| Bartonella | Recombinant Proteins | | | | | | |
|---|---|---|---|---|---|---|---|
| Species | htRA | SucB | N1pD | P35 | P31 | P26 | P17 |
| Bartonella | 1 | 5 | 9 | 13 | 17 | 27 | 37 |
| henselae | | | | | | | |
| Bartonella | 2 | 6 | 10 | 14 | 18 | 28 | 38 |
| quintana | | | | | | | |
| Bartonella | 3 | 7 | 11 | 15 | 19 | 29 | 39 |
| vinsonii | | | | | | | |
| Bartonella | 4 | 8 | 12 | 16 | 20 | 30 | 40 |
| elizabethae | | | | | | | |
| Bartonella | | | | | 21 | 31 | |
| bacilliformis | | | | | | | |
| Bartonella | | | | | 22 | 32 | |
| clarridgeiae | | | | | | | |
| Bartonella | | | | | 23 | 33 | |
| grahamii | | | | | | | |
| Bartonella | | | | | 24 | 34 | 41 |
| koehlerae | | | | | | | |
| Bartonella | | | | | 25 | 35 | |
| rochalimae | | | | | | | |
| Bartonella | | | | | 26 | 36 | |
| washoensis | | | | | | | |

A set of 61 left over decoded human sera, which included 31 samples that had been previously tested by Bartonella Western Blots IgM and IgG panels, and 30 serum samples, which included samples positive for antibodies to other tick-borne pathogens and controls, were tested using Bartonella IgM and IgG ImmunoBlots using the protocol described by Liu S et al., “Pilot Study of Immunoblots with Recombinant Borrelia burgdorferi Antigens for Laboratory Diagnosis of Lyme Disease,” Healthcare (Basel) 2018 Aug. 14; 6(3):99. doi: 10.3390/healthcare6030099.

Bartonella antigen strips were incubated with human sera for one hour at room temperature and washed three times to remove unbound serum. The washed strips were then incubated, for one hour, with alkaline phosphatase-conjugated goat anti-human IgG at 1:10,000 dilution, or IgM at 1:6000 dilution. After three washes, bands were visualized by reaction with 5-bromo-4-chloro-3-indolylphosphatenitro-blue tetrazolium. The strips were dried and read manually.

### Interpretation and Reporting

The Bartonella IgG and IgM ImmunoBlot Tests are qualitative assays that detects Bartonella specific IgG and IgM antibodies, respectively, in human serum. Recombinant Bartonella antigens were sprayed at specific positions onto nitrocellulose membrane and cut into strips. These strips were used to detect Bartonella specific antibodies in human serum. If the sample was positive for Bartonella antibodies, speciation was done to the following Bartonella species. Bartonella henselae, Bartonella quintana, Bartonella vinsonii, Bartonella elizahethae, and Bartonella sp.

The human serum was identified as positive for Bartonella antibodies if at least one band from each of the two following two groups was detected using IgM- or IgG-class secondary antibodies:
Group 1: SucB, NIpD, HTRA
Group 2: P35, P31, P26 and P17 are present or at least one band from each of the
following groups: SucB, NIpD and HTRA are present.

The test was indeterminate if two bands from Group 1 and only one band from Group 2 were detected using IgM- or IgG-class secondary antibodies.

Alternatively, the human serum was identified as positive for Bartonella antibodies if at least one band from each of SucB, N1pD, and HTRA was detected using IgM- or IgG-class secondary antibodies.

The test was negative if it exhibited any profile not meeting the foregoing positive or indeterminate criteria.

For a positive Bartonella Immunoblot, speciation was analyzed as follows:
positive for Bartonella henselae if at least one Bartonella henselae band, from at least one of P35, P31, and P26, detected using IgM- or IgG-class secondary antibodies;
positive for Bartonella quintana if at least one Bartonella quintana band, from at least one of P35, P31, and P26, detected using IgM- or IgG-class secondary antibodies;
positive for Bartonella vinsonii if at least one Bartonella vinsonii band, from at least one of P35, P31, and P26, detected using IgM- or IgG-class secondary antibodies; and
positive for Bartonella elizabethae if at least one Bartonella elizabethae band, from at least one of P35, P31, and P26, detected using IgM- or IgG-class secondary antibodies.

The human serum was identified as Bartonella sp. positive if the human serum was determined to be positive for Bartonella, but the foregoing speciation would not be determined due to presence of bands from multiple of the foregoing species.

### Limitations

A positive result suggested exposure to Bartonella. For diagnostic purposes, Immunoblot test results should be used in conjunction with clinical symptoms and other evidence available to the diagnosing physician. If the test result is indeterminate, testing with another method or retesting in 6-8 weeks is recommended.

### Results

FIG. 1 presents images showing results of IgM- and IgG-class Bartonella Immunoblots used to test nine human samples suspected of Bartonella infection.

FIG. 2A-B presents comparison images of Bartonella Western Blots (FIG. 2A) with Bartonella Immunoblots (FIG. 2B).

Results are summarized in Tables 4 and 5. As shown below in Table 4, of the 17 samples positive by Western Blots, 16 were also positive by the Immunoblots described above. Of the 6 Western Blot indeterminant samples, 5 were also indeterminant by the Immunoblots described above. All 8 Western Blot negative samples were negative by Immunoblot. As shown below in Table 5, of the 30 samples tested for specificity, none were positive by the Bartonella Immunoblot. Based on this data, the sensitivity of the Immunoblots was 94% as compared to Bartonella Western Blots, and the specificity is 100%.

TABLE 4
Performance of Bartonella ImmunoBlots (IB) as
compared to Bartonella Western Blots (WB).

| IgM | | | | | IgG | | | | | IgM and/or IgG | | | | |
|---|---|---|---|---|---|---|---|---|---|---|---|---|---|---|
| | N | (+) | IND | (−) | | N | (+) | IND | (−) | | N | (+) | IND | (−) |
| IB | 31 | 10 | 4 | 21 | IB | 31 | 13 | 5 | 18 | IB | 31 | 16 | 5 | 10 |
| WB | 31 | 10 | 2 | 21 | WB | 31 | 14 | 6 | 17 | WB | 31 | 17 | 6 | 8 |

TABLE 5
Specificity study.

| Sample Type (Positive | | Bartonella | Bartonella |
|---|---|---|---|
| for antibodies to) | N | Western Blot | ImmunoBlot |
| Borrelia burgdorferi (BB) | 5 | 0 | 0 |
| BB + Tick-Borne Relapsing | 2 | 0 | 0 |
| Fever Borrelia | | | |
| BB + Babesia | 3 | 0 | 0 |
| BB + Anaplasma phagocytophilia | 1 | 0 | 0 |
| Babesia | 7 | 0 | 0 |
| Controls | 12 | 0 | 0 |

TABLE 6
Sensitivity study showing sensitivity of Bartonella ImmunoBlots
is superior to Bartonella Western Blots in clinical samples.

| | | Bartonella | | Bartonella | |
|---|---|---|---|---|---|
| | Collection | Western Blot | | ImmunoBlot | |
| Subject | Date | IgM | IgG | IgM | IgG |
| JP | Sep. 15, 2020 | Neg | Neg | BE | BE/BV |
| GR | Aug. 11, 2020 | Neg | Bart sp./BE | Neg | BE/BV |
| GG | Jul. 13, 2020 | Bart sp. | Neg | BE/BH | Neg |
| AE | Oct. 15, 2020 | Neg | Neg | BE | Neg |

BE—Bartonella elizabethae; BV—Bartonella vinsonii; BH—Bartonella henselae; BQ—Bartonella quintana; Bart sp.—Bartonella species.

### Discussion and Conclusion

On a Western Blot, antigens are separated by size. Therefore, there can be multiple proteins at any given position on the blot. Because of that, there can be false positive results on Western Blots. In the case of ImmunoBlots, individual antigens are deposited (e.g., sprayed) at specific positions and, thus, there is no ambiguity in reading the blots. Based on the data, the Bartonella ImmunoBlots have the specificity and sensitivity for clinical use.

### EQUIVALENTS

Although several aspects of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific aspects of the disclosure described herein. It is, therefore, to be understood that the foregoing aspects are presented by way of example only and that, within the scope of the appended claims and equivalents thereto: the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1. A diagnostic panel comprising individually identifiable labelled, tagged, or bound amino acid sequences comprising SEQ ID NOs: 1-41, and variants thereof that retain the immunological binding profile of the corresponding non-variant.

2. The diagnostic panel of claim 1, wherein the amino acid sequences are bound to one or more substrates selected from the group consisting of nitrocellulose, nylon, polyvinylidene difluoride (PVDF), magnetic beads, agarose, plastic, and metal.

3. The diagnostic panel of claim 1, wherein the amino acid sequences comprise one or more labels selected from the group consisting of stable isotopes, mass tags, and fluorophores.

4. The diagnostic panel of claim 1, wherein the amino acid sequences comprise one or more peptide sequence tags selected from the group consisting of affinity tags, chromatography tags, epitope tags, enzyme conjugate tags, fluorescence tags, and combinations thereof.

5. A method for detecting infection by one or more Bartonella species, the method comprising:
contacting a biological sample, obtained from a subject suspected of having a Bartonella infection, with a diagnostic panel of claim 1, wherein the biological sample contains at least one of IgM-, IgG-, and IgA-class antibodies; and
determining the biological sample is positive for Bartonella infection when:
a positive immunobinding reaction with IgM-, IgG-, or IgA-class antibodies is detected for at least one of SEQ ID NOs: 1-12 and at least one of SEQ ID NOs: 13-41, or
a positive immunobinding reaction with IgM-, IgG-, or IgA-class antibodies is detected for at least one of SEQ ID NOs: 1-4, at least one of SEQ ID NOs: 5-8, and at least one of SEQ ID NOs: 9-12.

6. The method of claim 5, wherein the amino acid sequences are unlabelled, untagged, and unbound.

7-10. (canceled)

11. The method of claim 5, wherein the immunobinding of IgM-class antibodies is detected through the use of an anti-human IgM antibody linked to a detectable moiety; the immunobinding of IgG-class antibodies is detected through the use of an anti-human Ig antibody linked to a detectable moiety; and the immunobinding of IgA-class antibodies is detected through the use of an anti-human IgA antibody linked to a detectable moiety.

12-16. (canceled)

17. The method of claim 5, further comprising, in response to determining the biological sample is positive for the Bartonella infection, treating the subject for the Bartonella infection.

18. The method of claim 5, further comprising:
determining the biological sample is positive for Bartonella henselae infection when a positive immunobinding reaction with IgM-, IgG-, or IgA-class antibodies is detected for at least one of SEQ ID NOs: 13, 17, and 27;
determining the biological sample is positive for Bartonella quintana infection when a positive immunobinding reaction with IgM-, IgG-, or IgA-class antibodies is detected for at least one of SEQ ID NOs: 14, 18, and 28;
determining the biological sample is positive for Bartonella vinsonii infection when a positive immunobinding reaction with IgM-, IgG-, or IgA-class antibodies is detected for at least one of SEQ ID NOs: 15, 19, and 29; and
determining the biological sample is positive for Bartonella elizabethae infection when a positive immunobinding reaction with IgM-, IgG-, or IgA-class antibodies is detected for at least one of SEQ ID NOs: 16, 20, and 30.

19. The method of claim 18, further comprising, in response to determining the biological sample is positive for the Bartonella henselae infection, treating the subject for the Bartonella henselae infection; in response to determining the biological sample is positive for the Bartonella quintana infection, treating the subject for the Bartonella quintana infection; in response to determining the biological sample is positive for the Bartonella vinsonii infection, treating the subject for the Bartonella vinsonii infection; and in response to determining the biological sample is positive for the Bartonella elizabethae infection, treating the subject for the Bartonella elizabethae infection.

20-22. (canceled)

23. A diagnostic panel comprising individually identifiable labelled, tagged, or bound amino acid sequences comprising 1, 2, 3, or 4 of SEQ ID NOs: 1-4; 1, 2, 3, or 4 of SEQ ID NOs: 5-8, and 1, 2, 3, or 4 of SEQ ID NOs: 9-12, and variants thereof that retain the immunological binding profile of the corresponding non-variant.

24. The diagnostic panel of claim 23, comprising individually identifiable labelled, tagged, or bound amino acid sequences comprising SEQ ID NOs: 1-12 and variants thereof that retain the immunological binding profile of the corresponding non-variant.

25-27. (canceled)

28. A method for detecting infection by one or more Bartonella species, the method comprising:
contacting a biological sample, obtained from a subject suspected of having a Bartonella infection, with a diagnostic panel of claim 23, wherein the biological sample is determined to be positive for Bartonella infection when a positive immunobinding reaction with IgM-, IgG-, or IgA-class antibodies is detected for at least one of SEQ ID NOs: 1-4, at least one of SEQ ID NOs: 5-8, and at least one of SEQ ID NOs: 9-12.

29. The method of claim 28, wherein the diagnostic panel comprises amino acid sequences SEQ ID NOs: 1-12; and wherein the biological sample is determined to be positive for Bartonella infection when a positive immunobinding reaction with IgM-, IgG-, or IgA-class antibodies is detected for at least one of SEQ ID NOs: 1-4, at least one of SEQ ID NOs: 5-8, and at least one of SEQ ID NOs: 9-12.

30-34. (canceled)

35. The method of claim 28, wherein the immunobinding of IgM-class antibodies is detected through the use of an anti-human IgM antibody linked to a detectable moiety; the immunobinding of IgG-class antibodies is detected through the use of an anti-human Ig antibody linked to a detectable moiety; and the immunobinding of IgA-class antibodies is detected through the use of an anti-human IgA antibody linked to a detectable moiety.

36-40. (canceled)

41. The method of claim 28, further comprising, in response to determining the biological sample is positive for the Bartonella infection, treating the subject for the Bartonella infection.

42. A diagnostic panel comprising individually identifiable labelled, tagged, or bound amino acid sequences comprising SEQ ID NOs: 13-20 and 27-30, and variants thereof that retain the immunological binding profile of the corresponding non-variant.

43-45. (canceled)

46. A method for detecting species-specific infection by Bartonella henselae, Bartonella quintana, Bartonella vinsonii, and/or Bartonella elizabethae, the method comprising:
contacting a biological sample, obtained from a subject determined to have a Bartonella infection, with a diagnostic panel of claim 42, wherein the biological sample contains at least one of IgM-, IgG-, and IgA-class antibodies;
determining the biological sample is positive for Bartonella henselae infection when a positive immunobinding reaction with IgM-, IgG-, or IgA-class antibodies is detected for at least one of SEQ ID NOs: 13, 17, and 27;
determining the biological sample is positive for Bartonella quintana infection when a positive immunobinding reaction with IgM-, IgG-, or IgA-class antibodies is detected for at least one of SEQ ID NOs: 14, 18, and 28;
determining the biological sample is positive for Bartonella vinsonii infection when a positive immunobinding reaction with IgM-, IgG-, or IgA-class antibodies is detected for at least one of SEQ ID NOs: 15, 19, and 29; and
determining the biological sample is positive for Bartonella elizabethae infection when a positive immunobinding reaction with IgM-, IgG-, or IgA-class antibodies is detected for at least one of SEQ ID NOs: 16, 20, and 30.

47-51. (canceled)

52. The method of claim 46, wherein the immunobinding of IgM-class antibodies is detected through the use of an anti-human IgM antibody linked to a detectable moiety; the immunobinding of IgG-class antibodies is detected through the use of an anti-human Ig antibody linked to a detectable moiety; and the immunobinding of IgA-class antibodies is detected through the use of an anti-human IgA antibody linked to a detectable moiety.

53-57. (canceled)

58. The method of claim 46, further comprising, in response to determining the biological sample is positive for the Bartonella henselae infection, treating the subject for the Bartonella henselae infection: in response to determining the biological sample is positive for the Bartonella quintana infection, treating the subject for the Bartonella quintana infection: in response to determining the biological sample is positive for the Bartonella vinsonii infection, treating the subject for the Bartonella vinsonii infection; and in response to determining the biological sample is positive for the Bartonella elizabethae infection, treating the subject for the Bartonella elizabethae infection.

59-61. (canceled)
